(19) United States
(12) Patent Application Publication
(10) Pub. No.: US 2006/0040904 A1
(43) Pub. Date: Feb. 23, 2006

(54) Vaginal cream compositions, kits thereof and methods of using thereof

(76) Inventors: Salah U. Ahmed, New City, NY (US); Charles E. Diliberti, Montclair, NJ (US); Carole S. Ben-Maimon, Merion, PA (US); Howard Hait, Wilmington, DE (US)

Correspondence Address: STERNE, KESSLER, GOLDSTEIN & FOX PLLC, 1100 NEW YORK AVENUE, N.W., WASHINGTON, DC, 20005, US

(21) Appl. No.: 10/919,529

(22) Filed: Aug. 17, 2004

(51) Int. Cl. A61K 31/56

(52) U.S. Cl. 514/182

(57) ABSTRACT

The present invention is directed to pharmaceutical vaginal cream compositions comprising a conjugated estrogen and a stabilizer. The present invention is also directed to a method of treating a menopausal condition in a female in need thereof, said method comprising vaginally administering a pharmaceutical vaginal cream composition comprising a conjugated estrogen twice per week for at least 2 weeks.

### BACKGROUND OF THE INVENTION

1. Field of the Invention

The present invention is directed to pharmaceutical vaginal cream compositions comprising a conjugated estrogen and a stabilizer. The present invention is also directed to a method of treating a menopausal condition in a female in need thereof, said method comprising vaginally administering a pharmaceutical vaginal cream composition comprising a conjugated estrogen twice per week for at least 2 weeks.

2. Background Art

Decreased circulating estradiol levels occur with senescence of the ovaries and reduced follicular development, and cause characteristic symptoms at menopause (Bachmann, G. A. and Nevadunsky, N. S., Am. Fam. Phys. 61:3090-3096 (2000); Beers, M. R. and Berkow, R., eds., “Gynecology and Abstetrics,” in The Merck Manual of Diagnosis and Therapy, 17th Edition, Merck Research Laboratories, Whitehouse Station, N.J., 1942-1944 (1999)). Common symptoms include hot flashes, menstrual irregularities, night sweats, chills, insomnia, paresthesias, palpitations, tachycardia, cold hands and feet, headache, anxiety, dizziness, nervousness, depression, irritability, impaired cognition, diminished libido, fatigue, gastrointestinal symptoms, and atrophic vaginitis (Bachmann, G. A. and Nevadunsky, N. S., Am. Fam. Phys. 61:3090-3096 (2000); Beers, M. R. and Berkow, R., eds., “Gynecology and Abstetrics,” in The Merck Manual of Diagnosis and Therapy, 17th Edition, Merck Research Laboratories, Whitehouse Station, N.J., 1942-1944 (1999); Semmens, J. P. and Wagner, G., J. Am. Med. Assoc. 248:445-448 (1982); Bachmann, G. A., Maturitas 22 (Suppl.):S1-S5 (1995); Greendale, G. A., and Judd, H. L., J. Am. Geriatr. Soc. 41:426-436 (1993); Nilsson, K., et al., Maturitas 21:51-56 (1995)). Symptoms are frequently severe enough to cause women to seek treatment and may persist for several years during perimenopause and/or post-menopause.

Estrogen deprivation causes profound changes in the genitourinary tract, and up to 40% of postmenopausal women have symptoms associated with these changes (Greendale, G. A., and Judd, H. L., J. Am. Geriatr. Soc. 41:426-436 (1993)). A lack of vaginal lubrication and frequent vaginal infections are experienced by over 50% of post-menopausal women (Rosen, R., et al., J. Sex & Marital Therapy 19:171-188 (1993); Bachmann, G. A., Maturitas 22 (Suppl.):S1-S5 (1995)). The vaginal mucosa and vulvar skin become thinner, the labia flatten and shrink, and the clitoris, uterus, and ovaries decrease in size (Beers, M. R. and Berkow, R., eds., “Gynecology and Abstetrics,” in The Merck Manual of Diagnosis and Therapy, 17th Edition, Merck Research Laboratories, Whitehouse Station, N.J. (1999), pp. 1942-1944). Vaginal pH increases from the normal 3.5-4.0 (which favors lactobacilli) to 6.0-8.0 (which favors pathogenic organisms) (Pandit, L., and Ouslander, J. G., Am. J. Med. Sci. 314:228-231 (1997); Semmens, J. P. and Wagner, G., J. Am. Med. Assoc. 248:445-448 (1982)). Decreased pelvic muscle tone leads to urinary frequency, urgency, and incontinence (Bachmann, G. A., Maturitas 22 (Suppl.):S1-S5 (1995)). Endocervical glandular tissue becomes less active and mucus secretion decreases (Bachmann, G. A., Maturitas 22 (Suppl.):S1-S5 (1995)). The vaginal epithelium becomes dry and atrophic, which causes inflammation, discomfort, itching, and dyspareunia. The vagina becomes less distensible and elastic and is easily traumatized (Bachmann, G. A. and Nevadunsky, N. S., Am. Fam. Phys. 61:3090-3096 (2000)). Cytologic examination of vaginal mucosa shows an increased proportion of parabasal cells and a decreased proportion of superficial cells; e.g., a calculated Maturation Index score <55 (Pandit, L., and Ouslander, J. G., Am. J. Med. Sci. 314:228-231 (1997); Bachmann, G. A. and Nevadunsky, N. S., Am. Fam. Phys. 61:3090-3096 (2000); Nilsson, K., et al., Maturitas 21:51-56 (1995)). Vaginal ultrasonography of the uterine lining will typically demonstrate endometrium thinning to ≦5 mm, signifying decreased estrogen stimulation (Osmers, R., et al., Lancet 335:1569-1571 (1990)).

Estrogen therapy (ET) or hormone therapy (HT), if not contraindicated, is the treatment of choice for postmenopausal women with urogenital atrophy (Willhite, L. A. and O'Connell, M. B., Pharmacotherapy 21:464-480 (2001); Rigg., L. A., Int. J. Fertil. 31:29-34 (1986)). Various forms of HT have been shown to effectively manage menopausal signs and symptoms, including those associated with vaginal atrophy (Cardozo, I., et al., Obstet. Gynocol. 92:722-727 (1988); Beers, M. R. and Berkow, R., eds., “Gynecology and Abstetrics,” in The Merck Manual of Diagnosis and Therapy, 17th Edition, Merck Research Laboratories, Whitehouse Station, N.J., 1942-1944 (1999); Greendale, G. A., and Judd, H. L., J. Am. Geriatr. Soc. 41:426-436 (1993); Semmens, J. P. and Wagner, G., J. Am. Med. Assoc. 248:445-448 (1982); Bachmann, G. A., Maturitas 22 (Suppl.):S1-S5 (1995); Bachmann, G. A. and Nevadunsky, N. S., Am. Fam. Phys. 61:3090-3096 (2000); Nilsson, K., et al., Maturitas 21:51-56 (1995); Osmers, R., et al., Lancet 335:1569-1571 (1990); Rigg., L. A., Int. J. Fertil. 31:29-34 (1986); Marx, P., et al., Maturitas 47:47-54 (2004)). Estrogen therapy decreases vaginal pH (Bachmann, G. A. and Nevadunsky, N. S., Am. Fam. Phys. 61:3090-3096 (2000)), thickens and revascularizes the vaginal epithelium (Bachmann, G. A. and Nevadunsky, N. S., Am. Fam. Phys. 61:3090-3096 (2000)), increases the number of superficial cells (thereby increasing the Maturation Index) (Pandit, L., and Ouslander, J. G., Am. J. Med. Sci. 314:228-231 (1997)), and rapidly reverses vaginal atrophy (Bachmann, G. A. and Nevadunsky, N. S., Am. Fam. Phys. 61:3090-3096 (2000)).

A number of therapeutic regimens for estrogen replacement therapy are known, although many of these regimens comprise oral or transdermal administration of estrogens. Administration of conjugated equine estrogens, estradiol, and estriol vaginal creams has been shown to restore vaginal cytology to a premenopausal state and to improve urogenital atrophy (Willhite, L. A. and O'Connell, M. B., Pharmacotherapy 21:464-480 (2001)). The cyclic administration of conjugated estrogens daily for three weeks followed by one week off has been proposed (Premarin® Vaginal Cream package insert, revised Apr. 28, 2004, Wyeth Pharmaceuticals, Inc., Philadelphia, Pa.). However, results of the Women's Health Initiative (WHI) Study led to FDA recommendations that women receiving estrogen therapy be exposed to the lowest effective dose for the shortest duration of treatment (Hulley and Grady, J. Am. Med. Assoc. 291:1769-71 (2004)). Thus, the use of a less frequent dosing regimen of locally administered intravaginal estrogen therapy for the treatment of vulvovaginal atrophy has particular appeal.

### BRIEF SUMMARY OF THE INVENTION

The present invention is directed to pharmaceutical vaginal cream compositions comprising a conjugated estrogen and a stabilizer. The present invention is also directed to a method of treating a menopausal condition in a female in need thereof, the method comprising vaginally administering a pharmaceutical vaginal cream composition comprising a conjugated estrogen twice per week for at least 2 weeks.

### BRIEF DESCRIPTION OF THE DRAWINGS/FIGURES

FIG. 1 provides the mean pharmacokinetic profile for baseline-adjusted conjugated estrone after administration of the vaginal cream composition of Formulation B as described herein. FIG. 1A corresponds to the administration regimen termed Treatment A. FIG. 1B corresponds to the administration regimen termed Treatment B. FIG. 1C corresponds to the administration regimen termed Treatment C.

FIG. 2 provides the mean pharmacokinetic profile for baseline-adjusted estrone after administration of the vaginal cream composition of Formulation B as described herein. FIG. 2A corresponds to the administration regimen termed Treatment A. FIG. 2B corresponds to the administration regimen termed Treatment B. FIG. 2C corresponds to the administration regimen termed Treatment C.

FIG. 3 provides the mean pharmacokinetic profile for baseline-adjusted 17β-estradiol after administration of the vaginal cream composition of Formulation B as described herein. FIG. 3A corresponds to the administration regimen termed Treatment A. FIG. 3B corresponds to the administration regimen termed Treatment B. FIG. 3C corresponds to the administration regimen termed Treatment C.

FIG. 4 provides the mean pharmacokinetic profile for conjugated equilin after administration of the vaginal cream composition of Formulation B as described herein. FIG. 4A corresponds to the administration regimen termed Treatment A. FIG. 4B corresponds to the administration regimen termed Treatment B. FIG. 4C corresponds to the administration regimen termed Treatment C.

FIG. 5 provides the mean pharmacokinetic profile for equilin after administration of the vaginal cream composition of Formulation B as described herein. FIG. 5A corresponds to the administration regimen termed Treatment A. FIG. 5B corresponds to the administration regimen termed Treatment B. FIG. 5C corresponds to the administration regimen termed Treatment C.

### DETAILED DESCRIPTION OF THE INVENTION

1. Composition Comprising an Estrogen and a Stabilizer

The present invention is directed to pharmaceutical vaginal cream compositions comprising a conjugated estrogen and a stabilizer. In some embodiments, the present invention is directed to a pharmaceutical vaginal cream composition comprising two or more conjugated estrogens and a stabilizer. In some embodiments, the present invention is also directed to a kit or an applicator comprising a vaginal cream composition comprising a conjugated estrogen and a stabilizer. In some embodiments, the present invention is also directed to a method of treating a menopausal condition in a female in need thereof, the method comprising administering a vaginal cream composition comprising a conjugated estrogen and a stabilizer. In some embodiments, the present invention is also directed to a method of treating a menopausal condition in a female in need thereof, the method comprising vaginally administering a pharmaceutical vaginal cream composition comprising a conjugated estrogen twice per week for at least 2 weeks. In some embodiments, the present invention is also directed to a method of treating a menopausal condition in a female in need thereof, the method comprising vaginally administering a pharmaceutical vaginal cream composition comprising a conjugated estrogen and a stabilizer, wherein the composition is vaginally administered (a) at least once daily for at least 7 consecutive days, then (b) twice per week for at least 2 weeks.

Various conjugated estrogens can be used. An estrogen is any of various natural steroids or synthetic steroids that stimulate the development of female secondary sex characteristics and promote the growth and maintenance of the female reproductive system; or any other compound that mimics the physiological effect of natural estrogens. The term “conjugated” as described herein refers to the sulfate ester, glucuronide ester, or mixed sulfate-glucuronide esters, of an estrogen. Pharmaceutically suitable salt forms of the conjugated esters can be used in the present invention. In some embodiments, the salt is a sodium, potassium, or 2-amino-2-(hydroxymethyl)-1,3-propanediol (Tris) salt.

In some embodiments, the composition of the present invention comprises a conjugated estrogen such as, but not limited to, sodium estrone sulfate, sodium equilin sulfate, sodium 17α-dihydroequilin sulfate, sodium 17β-dihydroequilin sulfate, sodium 17α-estradiol sulfate, sodium 17β-estradiol sulfate, sodium equilenin sulfate, sodium 17α-dihydroequilenin sulfate, sodium 17β-dihydroequilenin sulfate or combination thereof. In some embodiments, the composition of the present invention comprises a conjugated estrogen such as, but not limited to, sodium estrone sulfate, sodium equilin sulfate, sodium 17α-dihydroequilin sulfate, sodium 17β-dihydroequilin sulfate, sodium 17α-estradiol sulfate, sodium 17β-estradiol sulfate, sodium equilenin sulfate, sodium 17α-dihydroequilenin sulfate, sodium 17β-dihydroequilenin sulfate, Δ8,9-dehydroestrone sulfate or combination thereof. In some embodiments, the conjugated estrogen is sodium ethinyl estradiol sulfate. In some embodiments, the conjugated estrogen is a mixture of 9 estrogenic substances, such as, e.g., the mixture of estrogens found in Cenestin® tablets (Duramed Pharmaceuticals, Inc., Pomona, N.Y.; see Cenestin® prescribing information, revised February 2004). In some embodiments, the conjugated estrogen is a mixture of 10 estrogenic substances; e.g., the mixture of estrogens found in Enjuvia® (Endeavor Pharmaceuticals, Inc., Wilmington, N.C.; see Enjuvia® package insert, revised May 4, 2004).

In some embodiments, the composition of the present invention comprises conjugated estrogens, wherein the conjugated estrogens consist of a combination of sodium estrone sulfate, sodium equilin sulfate, sodium 17α-dihydroequilin sulfate, sodium 17β-dihydroequilin sulfate, sodium 17α-estradiol sulfate, sodium 17β-estradiol sulfate, sodium equilenin sulfate, sodium 17α-dihydroequilenin sulfate, and sodium 17β-dihydroequilenin sulfate. In some embodiments, the conjugated estrogens consist of a combination of sodium estrone sulfate, sodium equilin sulfate, sodium 17α-dihydroequilin sulfate, sodium 17β-dihydroequilin sulfate, sodium 17α-estradiol sulfate, sodium 17β-estradiol sulfate, sodium equilenin sulfate, sodium 17α-dihydroequilenin sulfate, sodium 17β-dihydroequilenin sulfate, and Δ8,9-dehydroestrone sulfate.

Various stabilizers can be used in the present invention. The term “stabilizer” refers to any substance that keeps the estrogen chemically stable. Alternatively, the term “stabilizer” refers to any substance that slows or retards the degradation or alteration of an estrogen. For example, a stabilizer can protect the estrogen from instability caused by light, moisture, heat, or oxidation. In some embodiments, the stabilizer is lipophilic. In some embodiments, the stabilizer is hydrophilic. In some embodiments, the stabilizer can prevent or retard the oxidation of the oil. In some embodiments, the stabilizer can be, but is not limited to, butylated hydroxyanisole (BHA), butylated hydroxytoluene (BHT), ascorbic acid and its esters, vitamin E and its esters, e.g., vitamin E acetate, sodium bisulfite, sodium metabisulfite, 3-dehydroshikimic acid (DHS), tocopherols and their esters, alkyl gallates, chelating agents, EDTA (ethylenediaminetetraacetic acid; edetate disodium), citric acid, benzyl alcohol, or combinations thereof. In some embodiments, the stabilizer can be edetate disodium, butylated hydroxyanisole, butylated hydroxytoluene, or combinations thereof.

In some embodiments, the composition of the present invention further comprises a pharmaceutically acceptable excipient. As used herein, “excipient” refers to a substance, or mixture of substances, that is used in the formulation of vaginal cream compositions to give desirable physical characteristics to the formulation. As used herein, the term “pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio. In some embodiments, the term “pharmaceutically acceptable” means approved by a regulatory agency of the Federal or a state government or listed in the U.S. Pharmacopeia or other generally recognized international pharmacopeia for use in animals, and more particularly in humans. Various pharmaceutically acceptable excipients can be used. In some embodiments, the pharmaceutically acceptable excipient can be, but is not limited to, a stiffening agent, an oil, a solvent, an emulsifier, a humectant, a buffering agent, a filler, an emollient, a stabilizer, or combinations thereof.

The term “stiffening agent” refers to a substance, or mixture of substances, added to make a vaginal cream composition more viscous at room temperature. In some embodiments, a stiffening agent is any substance that promotes formation of a formulation having a semi-solid consistency. The stiffening agent can be hydrophilic (e.g., carbopol, carboxymethylcellulose, hydroxypropylmethylcellulose, alginate, polyethylene glycol). In some embodiments, the stiffening agent has low hydrophilic-lipophilic balance (HLB). In some embodiments, the HLB value is less than 7. In some embodiments, the HLB value is less than 5. In some embodiments, the HLB value is about 4. Examples of suitable stiffening agents include, but are not limited to, hydrogenated vegetable oil, cetyl alcohol, cetyl esters wax, microcrystalline wax, paraffin, stearyl alcohol, lauryl alcohol, myristal alcohol, cetostearyl alcohol, white wax, yellow wax, beeswax, candelilla wax, cotton wax, carnauba wax, bayberry wax, rice-bran wax, and combinations thereof. In some embodiments, the stiffening agent is a mixture of cetyl esters wax, cetyl alcohol, and beeswax.

The term “oil” refers to any pharmaceutically acceptable hydrophobic liquid. In some embodiments, an oil is an ester of glycerol (1,2,3-propanetriol) and fatty acids. Generally, the fatty acid hydrocarbon chains each contain greater than 8 carbons. In some embodiments, each hydrocarbon chain can contain from about 12 to about 36 carbon atoms. In some embodiments, the hydrocarbon chains can contain a variety of functional groups. In some embodiments, the hydrocarbon chain can be branched. In some embodiments, the hydrocarbon chains are unsaturated or polyunsaturated. In some embodiments, the hydrocarbon chains are saturated. The degree of saturation can affect the physical state, for example viscosity, of the oil. In some embodiments, the oil can be, but is not limited to, vegetable, nut, and seed oils (e.g., almond oil, castor oil, coconut oil, corn oil, cotton seed oil, jojoba oil, linseed oil, grape seed oil, rape seed oil, mustard oil, olive oil, palm and palm kernel oil, peanut oil, safflower oil, sesame oil, soybean oil, sunflower-seed oil, crambe oil, wheat germ oil, and cocoa butter), hydrocarbon and petroleum oils (e.g., petrolatum, mineral oil, and liquid paraffin). In some embodiments, the term “oil” refers to higher fatty acids (e.g., lauric acid, myristic acid, palmitic acid, stearic acid, behenic acid, oleic acid, 12-hydroxystearic acid, undecylenic acid, tall acid, lanolin fatty acid, isostearic acid, linoleic acid, and linolenic acid) and combinations thereof. In some embodiments, the oil is not an ester of glycerol, e.g., mineral oil and silicone oil.

The term “solvent” refers to any substance capable of dissolving or dispersing one or more of the conjugated estrogens or the excipients of the present invention. The solvent can be aqueous or non-aqueous. In some embodiments, the solvent is hydrophilic, and is 10% to 75% by weight, or 20% to 60% by weight, of the total composition. In some embodiments, the solvent is lipophilic, and is 20% to 60% by weight, or 25% to 50% by weight, of the total composition. In some embodiments, the solvent is water, a polyol (e.g., glycerol) or combinations thereof. In some embodiments, the solvent is an oil as described above.

The term “emulsifier” refers to any substance that promotes formation and stabilization of an emulsion or suspension. In some embodiments, the emulsifier includes, but is not limited to, sodium lauryl sulfate, propylene glycol monostearate, methyl stearate, glyceryl monostearate, and combinations thereof.

The term “humectant” refers to any substance that promotes retention of moisture in the composition of the present invention. In some embodiments, the humectant includes, but is not limited to, polyethylene glycol, propylene glycol, glycerin, polyol, polyol derivatives, and combinations thereof.

The term “buffering agent” refers to any substance capable of neutralizing both acids and bases and thereby maintaining the desired pH of the composition of the present invention. In some embodiments, the buffering agent affects the emulsifying properties. For example, different buffering agents can be provided to increase or decrease the emulsification of the conjugated estrogens or the excipients of the present invention. In some embodiments, the buffer can be, but is not limited to, Tris buffers (Tris EDTA (TE), Tris acetate (TAE), Tris phosphate (TPE), Tris glycine), phosphate buffers (e.g., sodium phosphate, potassium phosphate), bicarbonate buffers, acetate buffers (e.g., sodium acetate), ammonium buffers, citrate buffers, and derivatives and combinations thereof. In some embodiments, an organic acid buffer is used. In some embodiments, an acetate buffer, a phosphate buffer, or a citrate buffer can be used. In some embodiments, a zwitterionic buffer can be used. In some embodiments, the buffering agent is a phosphate buffer (e.g., sodium phosphate dibasic).

The pH of the composition of the invention can be physiologically compatible and/or sufficient to maintain stability of the composition. In some embodiments, the composition of the present invention can have a pH of 5.0 to 9.0, or a pH of 6.5 to 8.0.

As defined herein, an “emollient” is a substance that moisturizes and increases the pliability of the vaginal epithelium. In some embodiments, the emollient can be, but is not limited to, lanolin, isopropyl myristate, palmitate, oleyl alcohol, beeswax, mineral oil, silicone oil, or combinations thereof.

As defined herein, a “filler” is a substance used to give bulk to the composition without chemically reacting with the conjugated estrogens of the present invention. Fillers are known to those in the art, see e.g., Remington: The Science and Practice of Pharmacy, 20^{th} ed. (2000).

As defined herein, a “vaginal cream” is a semi-solid preparation suitable for application to the vaginal tract. In some embodiments, a vaginal cream can be a vaginal ointment, vaginal gel or vaginal emulsion. Various classes of vehicle bases can be used in the vaginal cream and are known to those in the art. For example, suitable vehicle bases include, but are not limited to, hydrocarbon bases or oleaginous bases, absorption bases, water-removable bases and water-soluble bases (Remington: The Science and Practice of Pharmacy, 20^{th} ed. (2000)). In some embodiments, the vehicle base is non-irritating, non-staining, stable, non-pH dependent and/or compatible with the conjugated estrogens of the present invention.

The amount of active agent or agents in a dosage form can vary. The exact dosage amount can be selected depending upon the needs of the female to which the active agent is being administered, as determined by a relevant person. In some embodiments, one of skill in the art can perform pharmacokinetic studies and use the results of the study to adjust the dosage amount for a female, or a group of females, to a suitable level. In some embodiments, one of skill in the art can determine an appropriate dosage amount based on varying dosage amounts and comparing to symptomatic relief. In some embodiments, appropriate animal studies may be performed to determine an appropriate dosage amount. A “relevant person” as used herein, includes, for example, a physician, physician assistant, nurse practitioner, pharmacist and customer service representative.

Various amounts of conjugated estrogens in the composition of the present invention can be present in a dosage form. In some embodiments, the composition of the present invention is in a dosage form, wherein the dosage form comprises 0.1 mg/dose to 3 mg/dose, or 0.3 mg/dose to 2.5 mg/dose of conjugated estrogens. As used herein, unless a specific estrogen is identified, amounts of “conjugated estrogens” refer to a summation of the amounts of three estrogens: sodium 17α-dihydroequilin sulfate, sodium estrone sulfate, and sodium equilin sulfate. In some embodiments, the composition of the present invention is in a dosage form, wherein the dosage form comprises 1.25 mg/dose of conjugated estrogens.

Various types of conjugated estrogens of the present invention can be present in the vaginal cream composition in varying amounts. In some embodiments, the percentage of the estrogens can be found within the ranges listed in Table 1.

TABLE 1

| Estrogens* | Range A | Range B | Range C | Range D |
|---|---|---|---|---|
| 17α-Estradiol | 0%-99% | 1%-20% | 2%-10% | 3.5%-7% |
| 17α- | 0%-99% | 5%-30% | 7%-25% | 10%-19% |
| Dihydroequilin | | | | |
| 17β- | 0%-99% | 0.1%-10% | 0.1%-5% | 0.5%-3.5% |
| Dihydroequilin | | | | |
| Estrone | 0%-99% | 25%-75% | 50%-65% | 51%-62% |
| Equilin | 0%-99% | 10%-50% | 15%-35% | 20%-31% |
| 17β-Estradiol | 0%-99% | 0%-5% | 0.1%-4% | 0.5%-2% |
| 17α- | 0%-99% | 0%-5% | 0.1%-4% | 0.2%-5% |
| Dihydroequilenin | | | | |
| 17β- | 0%-99% | 0%-5% | 0.05%-2% | 0.1%-1.5% |
| Dihydroequilenin | | | | |
| Equilenin | 0%-99% | 0%-15% | 0.1%-10% | 0.5%-6.5% |

*all estrogens reported as the sodium salts of 3-monosulfate esters

As shown in Table 1, various combinations and amounts of conjugated estrogens can be used in the present invention. In some embodiments, the composition of the present invention is in a dosage form, wherein the dosage form comprises 0.3 mg/dose to 2.5 mg/dose of conjugated estrogens, wherein the conjugated estrogens consist of (a) 3.5% to 7.0% by weight sodium 17α-estradiol sulfate; (b) 10.0% to 19.0% by weight sodium 17α-dihydroequilin sulfate; (c) 0.5% to 3.5% by weight sodium 17β-dihydroequilin sulfate; (d) 51.0% to 62.0% by weight sodium estrone sulfate; (e) 20.0% to 31.0% by weight sodium equilin sulfate; (f) 0.5% to 2.0% by weight sodium 17β-estradiol sulfate; (g) 0.2% to 5.0% by weight sodium 17α-dihydroequilenin sulfate; (h) 0.1% to 1.5% by weight sodium 17β-dihydroequilenin sulfate; and (i) 0.5% to 6.5% by weight sodium equilenin sulfate; or an amount of a mixture of conjugated estrogens that provides an estrogenic effect equivalent to that produced by the 0.3 mg to 2.5 mg of conjugated estrogens as defined in (a)-(i).

In some embodiments, the amount of a particular estrogen can be in the ranges specified in Table 2.

TABLE 2

| | Dosage Amounts (μg/dosage form) | | |
|---|---|---|---|
| Estrogen* | Range E | Range F | Range G |
| 17α-Estradiol | 0-2500 | 1.0-17.5 | 43.75-87.5 |
| 17α-Dihydroequilin | 0-2500 | 30-475 | 125-237.5 |
| 17β-Dihydroequilin | 0-2500 | 1.5-87.5 | 6.25-43.75 |
| Estrone | 0-2500 | 153-1550 | 637.5-775 |
| Equilin | 0-2500 | 60-775 | 250-387.5 |
| 17β-Estradiol | 0-2500 | 1.5-50 | 6.25-25 |
| 17α-Dihydroequilenin | 0-2500 | 0.6-125 | 2.5-62.5 |
| 17β-Dihydroequilenin | 0-2500 | 0.3-37.5 | 1.25-18.75 |
| Equilenin | 0-2500 | 1.5-162.5 | 6.25-81.25 |

*All estrogens reported as the sodium salts of the 3-monosulfate esters.

The ranges listed in Table 2 for each estrogen are mutually exclusive from the ranges for the other estrogens.

The listed estrogens, as well as other estrogens, vary in potency from each other. The ranges given above are for the specified estrogen, however if a different estrogen is employed, adjustments in the amount employed, based on the relative potency, can be made and are well known in the art. The correlations in potency between various estrogens are known. See, for example, EP 0 253 607, which is hereby incorporated in its entirety by reference. Equivalent concentrations of estrogens can be determined using either in vitro or in vivo assay methods (Kuhl, H., Drugs 51:188-215 (1996); Philibert, D., et al., Gynecol. Endocrinol. 13:316-326 (1999); and Lundeen, S., et al., J. Steroid Biochem. Molec. Biol. 78:137-143 (2001)). When in vitro receptor binding studies are performed to determine relative potency, the unconjugated forms of an estrogen should be used. See also, for example, Dickey, R. P., “Contraceptive Therapy,” OBG Management Supplement (October 2000), pp. 2-6. As described herein, the term “relative potency,” “equivalent amount,” or “amount equivalent to” can be determined by a method described by Mandel et. al. (J. Clin. Endocrinol. Metabol. 57:133-139 (1983)). Each of these documents is hereby incorporated by reference in its entirety.

2. Method of Treating Menopausal Conditions

The terms “treat” and “treatment” refer to both therapeutic treatment and prophylactic or preventative measures, wherein the object is to prevent, inhibit, reverse or slow down (lessen) an undesired physiological condition, disorder or disease, or obtain beneficial or desired clinical results. For purposes of this invention, beneficial or desired clinical results include, but are not limited to, alleviation of symptoms; diminishment of extent of condition, disorder or disease; stabilized (i.e., not worsening) state of condition, disorder or disease; delay in onset, or slowing, of condition, disorder or disease progression; amelioration of the condition, disorder or disease state, remission (whether partial or total); or enhancement or improvement of condition, disorder or disease. Treatment also includes, but is not limited to, eliciting a cellular response that is clinically significant, without excessive levels of side effects.

“Female” refers to any animal classified as a mammal which menstruates, including primates, e.g., humans. “Female” also refers to other nonhuman mammals, e.g., domestic and farm animals, and zoo, sports, and companion animals such as household pets and other domesticated animals such as, but not limited to, cattle, sheep, ferrets, swine, horses, rabbits, goats, dogs, cats and the like. In some embodiments, companion animals are dogs and cats.

The invention is directed to a method of treating a menopausal condition in a female in need thereof, the method comprising vaginally administering a pharmaceutical composition of the present invention. In some embodiments, the composition is administered once per day. In some embodiments, the composition is administered multiple times a day, for example, twice a day.

In some embodiments, the vaginal cream composition comprising a conjugated estrogen and a stabilizer is vaginally administered twice per week for at least 2 weeks. In some embodiments, the composition comprising a conjugated estrogen and a stabilizer is administered vaginally at least once. In some embodiments, the composition comprising a conjugated estrogen and a stabilizer is administered vaginally once daily for at least 7 consecutive days. In some embodiments, the vaginal cream composition comprising a conjugated estrogen and a stabilizer is vaginally administered (a) at least once daily for at least 7 consecutive days, then (b) twice per week for at least 2 weeks. In some embodiments, the composition comprising a conjugated estrogen and a stabilizer is vaginally administered (a) once daily for 7 consecutive days, then (b) twice a week for at least 2 weeks.

In some embodiments, the present invention is directed to a method of treating a menopausal condition in a female in need thereof, the method comprising vaginally administering a pharmaceutical vaginal cream composition comprising a conjugated estrogen, twice per week for at least 2 weeks. In some embodiments, the present invention is directed to a method of treating a menopausal condition in a female in need thereof, the method comprising vaginally administering a pharmaceutical vaginal cream composition comprising a conjugated estrogen vaginally (a) once daily for at least 2 consecutive days, then (b) twice per week for at least 2 weeks. In some embodiments the present invention is directed to a method of treating a menopausal condition in a female in need thereof, the method comprising vaginally administering a pharmaceutical vaginal cream composition comprising a conjugated estrogen (a) at least once daily for 7 consecutive days, then (b) twice per week for at least 2 weeks.

In some embodiments, the method of treatment can be divided into two stages. The “starter” stage encompasses daily vaginal administration of the vaginal cream comprising a conjugated estrogen. In some embodiments, the starter stage encompasses administration of the vaginal cream composition at least once daily for 7 consecutive days as described in (a) above. In some embodiments, the starter stage encompasses administration of the composition of the present invention daily for 2 to 13 consecutive days. In some embodiments, the starter stage encompasses administration of the composition of the present invention daily for 5 to 13 consecutive days. In some embodiments, the composition is administered once daily for 7 consecutive days. In some embodiments, treatment of a female by administering a starter stage is preferable for a female who has not recently been treated for a menopausal condition using any other hormone therapy. In some embodiments, treatment of a female by administering a starter stage is preferable for a female who has previously been on hormone therapy, but has stopped taking the therapy for such a time as to allow the vaginal cytology of the female to revert to a substantially post-menopausal state. In some embodiments, treatment of a female by administering a starter stage is preferable for a female whose vaginal epithelial cytology is in a menopausal state.

The “maintenance” stage encompasses administration of the vaginal cream twice per week for at least two weeks. In some embodiments, the maintenance stage follows the starter stage. In some embodiments, the maintenance stage comprises administering the composition of the present invention for two weeks. However, in some embodiments, the maintenance stage can be longer in duration. For example, the maintenance stage can continue until the menopausal condition being treated no longer requires treatment. In some embodiments, the maintenance stage continues for 2 weeks to 2 years. In some embodiments, the maintenance stage continues for 2 weeks to 1 year. In some embodiments, the maintenance continues for 3 weeks to 4 weeks. In some embodiments, the maintenance stage continues for 3.5 weeks. In some embodiments, treatment of a female by administering the maintenance stage without a starter stage is preferable for a female who has recently been treated for a menopausal condition using hormone therapy. In some embodiments, treatment of a female by administering the maintenance stage without a starter stage is preferable for a female whose vaginal epithelial cytology is in a premenopausal state.

The term “once daily” refers to administration of a composition of the present invention once during a 24 hour period. In some embodiments, the composition is administered once per day. In some embodiments, the composition is administered twice per day. In some embodiments, the composition is administered more than twice per day.

The present invention is suitable for treatment of various menopausal conditions. The term “menopausal condition” relates to conditions associated with menopause, or to the period of natural cessation of menstruation. Additionally, the term “menopausal condition” also relates to conditions related to peri-menopause, post menopause, or oophorectomized women. Thus, the term menopausal condition is not limited to females that are undergoing menopause, but also women who are undergoing peri-menopause or post-menopause, women who have been bilaterally oophorectomized, or women whose endogenous sex hormone production has been suppressed by pharmaceutical chemical compositions, e.g., GnRH agonists such as leuprolide-acetate sold under the tradename LUPRONE® (TAP Pharmaceutical Products, Inc) or goserilin acetate, sold under the tradename ZOLADEX® (AstraZeneca). The phrase “vaginal epithelial cytology is in a premenopausal state” refers to the state of a vaginal epithelial of a woman who has not yet entered perimenopause or menopause. The phrase “vaginal epithelial cytology is in a menopausal state” refers to the state of a vaginal epithelial of a woman who is in menopause and is not taking any form of hormone replacement therapy. The state of the vaginal epithelial can be evaluated as described by Mandel et al. (J. Clin. Endocrinol. Metabol. 57: 133-139 (1983)).

Various menopausal conditions can exist. In some embodiments, a menopausal condition can be, but is not limited to, vaginal dryness, pain during intercourse, increased risk of infections, inability to control urination (incontinence), increased frequency of urinary infections, vaginal atrophy, kraurosis vulvae, hot flashes and night sweats, fatigue, emotional changes (mood swings and changes in sexual interest), sleep disturbances (insomnia), drier skin and hair, increased growth of facial and body hair, aches and pains in the joints, headaches, palpitations (rapid, irregular heart beats), vaginal itching, osteoporosis, osteopenia, and generalized itching.

The vaginal composition of the present invention is administered vaginally by placing the vaginal cream composition comprising a conjugated estrogen in contact with the vaginal tract of the female being treated. In some embodiments, once the vaginal cream is administered, the estrogens act locally on the vaginal epithelial, i.e., non-systemically. In some embodiments, once the vaginal cream is administered, the estrogens act systemically on the female being treated. Thus, in some embodiments, administration of the vaginal cream composition of the present invention provides systemic treatment of a menopausal condition. In some embodiments, administration of the vaginal cream composition of the present invention provides both systemic and local treatment of a menopausal condition.

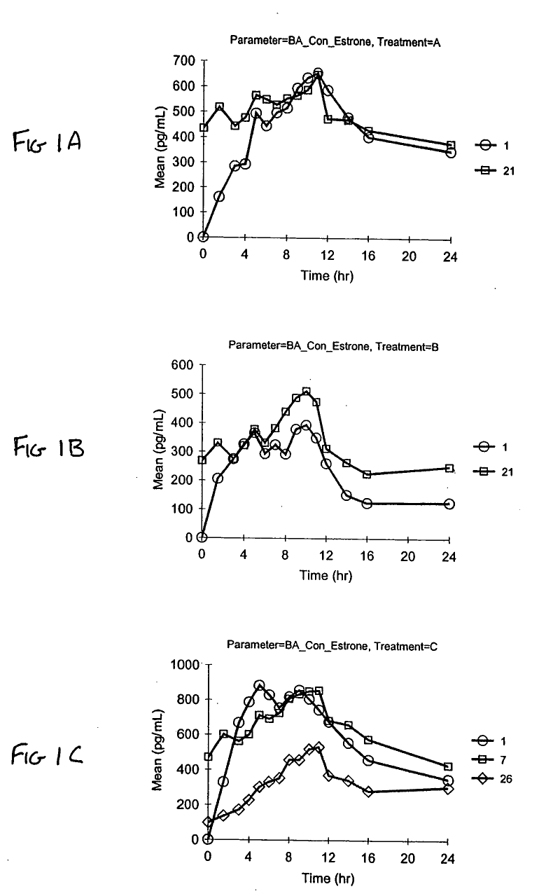

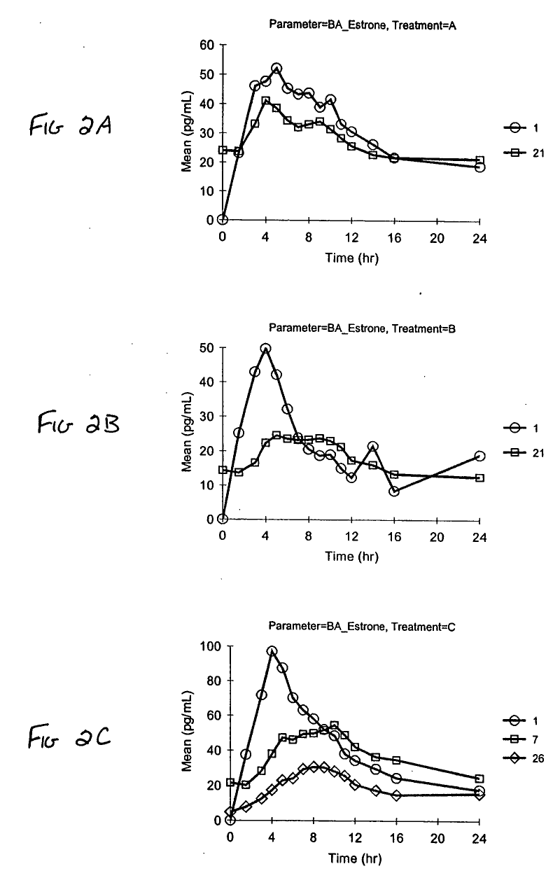

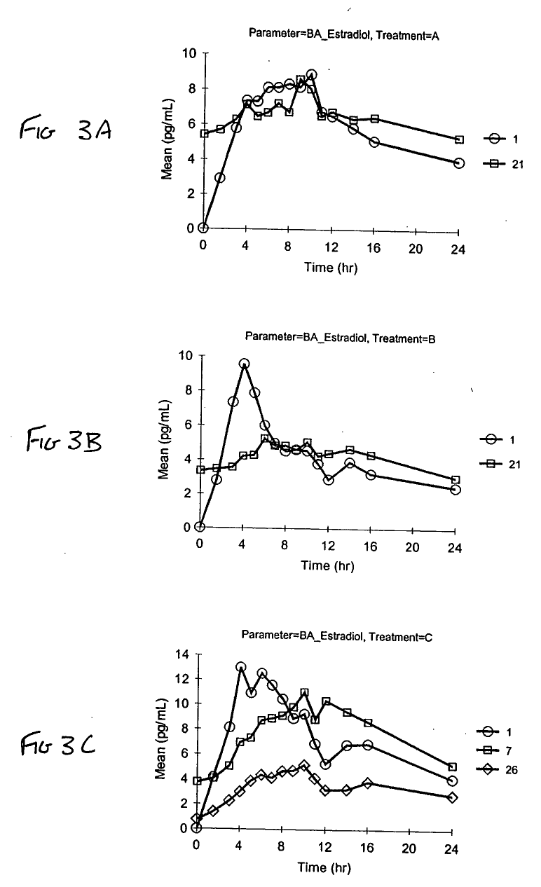

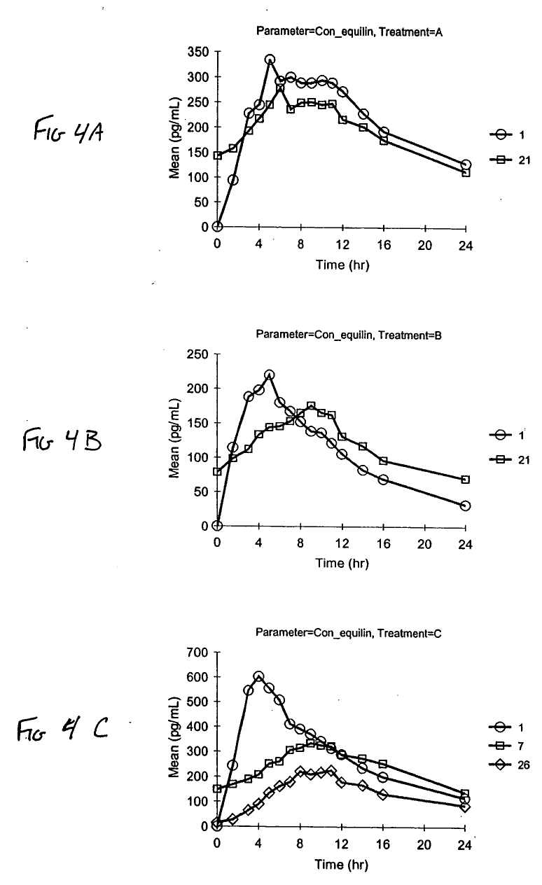

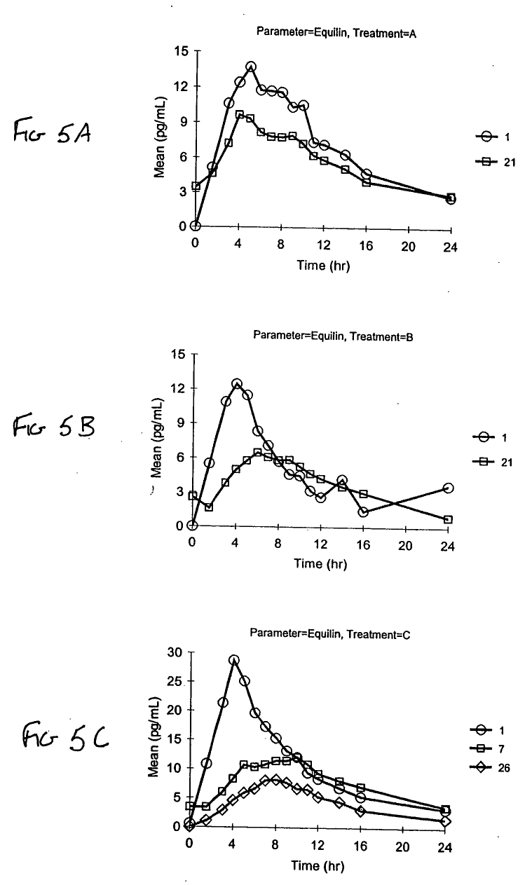

Administration of the composition of the present invention can produce a pulsatile pharmacokinetic delivery profile of estrogens. After administration of the composition of the present invention, an initial increase in the plasma concentration of estrogens occurs. Blood plasma concentration levels of estrogens then peak, after which there is a decrease in the plasma concentration of estrogens in the female being treated. Examples of pulsatile pharmacokinetic profiles are found in FIGS. 1-5. Upon administration of another dose of the composition of the present invention, the plasma concentration of estrogen again increases, peaks, and then decreases. The term “pulsatile pharmacokinetic profile” refers to the cyclic increase, peak, and decrease of plasma concentrations of estrogens. In some embodiments, the pulsatile pharmacokinetic profile is reduced upon repeated administration of the composition of the present invention. That is, the peak plasma concentration of estrogens is less than the peak plasma concentration of estrogens achieved by the previous administration of the composition of the present invention. While not bound by any hypothesis, this reduction in exposure after multiple dosings possibly can be attributed to the reduction in absorption through the vaginal epithelial due to the improvement of vaginal epithelium from the administration of the conjugated estrogens. However, after repeated administration of the estrogens, an essentially steady state pharmacokinetic profile is reached, resulting in a relatively constant pulsatile pharmacokinetic profile. That is, the peak at steady state following administration of the composition of the present invention remains relatively constant upon administration of additional doses of the composition of the present invention. In some embodiments, after two weeks, the pulsatile delivery of estrogens is at steady state. In some embodiments, after four weeks, the pulsatile delivery of estrogens is at steady state.

Various dosage amounts of the vaginal cream composition comprising a conjugated estrogen can be administered during, e.g., the 7 consecutive days of administration (a) to provide various plasma levels of estrogens in the female being treated. The minimum concentration (Cₘᵢₙ) as defined herein for the “at least daily for 7 consecutive days” regimen is the concentration of estrogens in the patient's plasma 48 hours after the seventh dose. The Cₘᵢₙ as defined herein for the “twice per week for at least two weeks” regimen is the concentration of the estrogens in the patient's plasma 48 hours after the thirteenth dose (7 daily doses and 6 twice weekly doses).

Suitable Cₘᵢₙ of the various estrogens are described in Table 3.

TABLE 3

| | At least daily for 7 consecutive | | | Twice per week for at least 2 | | |
|---|---|---|---|---|---|---|
| | days⁽¹⁾ (pg/ml) | | | weeks⁽²⁾ (pg/ml) | | |
| | Range A | Range B | Range C | Range A | Range B | Range C |
| baseline- | 20-400 | 36-305 | 152.5 ± 10% | 20-400 | 33-277 | 138.3 ± 10% |
| adjusted | | | | | | |
| conjugated | | | | | | |
| estrone | | | | | | |
| baseline- | 1-25 | 2-17 | 8.31 ± 10% | 1-25 | 1-14 | 6.6 ± 10% |
| adjusted | | | | | | |
| estrone | | | | | | |
| baseline- | 0-10 | 0-5 | 2.32 ± 10% | 0-5 | 0-1 | 0.46 ± 10% |
| adjusted | | | | | | |
| 17β- | | | | | | |
| estradiol | | | | | | |
| conjugated | 5-100 | 10-87 | 43.11 ± 10% | 1-50 | 4-40 | 19.86 ± 10% |
| equilin | | | | | | |
| equilin | 0-3 | 0-1 | 0.29 ± 10% | 0-3 | 0-0.5 | 0.1 ± 10% |

⁽¹⁾range determined 48 hours after the seventh dose of the at least daily for 7 consecutive days regimen.
⁽²⁾range determined 48 hours after the thirteenth dose of the twice per week for at least two weeks regimen.
All values are average (mean) values.

In some embodiments, 48 hours after the seventh dose during the (a) at least daily for 7 consecutive days of administration, the method of the present invention provides an average baseline-adjusted conjugated estrone Cₘᵢₙ of 36 pg/mL to 305 pg/mL. In some embodiments, 48 hours after the seventh dose during the (a) 7 consecutive days of administration, the method of the present invention provides an average baseline-adjusted estrone Cₘᵢₙ of 2 pg/mL to 17 pg/mL. In some embodiments, 48 hours after the seventh dose during the (a) 7 consecutive days of administration, the method of the present invention provides an average baseline-adjusted 17β-estradiol Cₘᵢₙ of 0 pg/mL to 5 pg/mL. In some embodiments, 48 hours after the seventh dose during the (a) 7 consecutive days of administration, the method of the present invention provides an average conjugated equilin Cₘᵢₙ of 10 pg/mL to 87 pg/mL. In some embodiments, 48 hours after the seventh dose during the (a) 7 consecutive days of administration, the method of the present invention provides an average equilin Cₘᵢₙ of 0 pg/mL to 1 pg/mL.

Various dosage amounts of the vaginal cream composition comprising a conjugated estrogen can be administered during the (b) twice per week for at least two weeks of administration to provide various plasma levels of estrogens in the female being treated. In some embodiments, 48 hours after the thirteenth dose during the (b) twice per week for at least two weeks of administration, the method of the present invention provides an average baseline-adjusted conjugated estrone Cₘᵢₙ of 33 pg/mL to 277 pg/mL. In some embodiments, 48 hours after the thirteenth dose during the (b) twice per week for at least two weeks of administration, the method of the present invention provides an average baseline-adjusted estrone Cₘᵢₙ of 1 pg/mL to 14 pg/mL. In some embodiments, 48 hours after the thirteenth dose during the (b) twice per week for at least two weeks of administration, the method of the present invention provides an average baseline-adjusted estradiol Cₘᵢₙ of 0 pg/mL to 1 pg/mL. In some embodiments, 48 hours after the thirteenth dose during the (b) twice per week for at least two weeks of administration, the method of the present invention provides an average conjugated equilin Cₘᵢₙ of 4 pg/mL to 40 pg/mL. In some embodiments, 48 hours after the thirteenth dose during the (b) twice per week for at least two weeks of administration, the method of the present invention provides an average equilin Cₘᵢₙ of 0 pg/mL to 0.5 pg/mL.

In some embodiments, the method of the present invention can provide to the female various plasma concentration versus time curves (pg/mL versus hours) which produce various average area under the curve (AUC₀₋₂₄) values for the various estrogens. In some embodiments, the AUC₀₋₂₄ values for the specified estrogens fall within the ranges listed in Table 4.

TABLE 4

| | At least daily for 7 consecutive | | | Twice per week for at least 2 | | |
|---|---|---|---|---|---|---|
| | days regimen⁽¹⁾ (pg hr/ml) | | | weeks regimen⁽²⁾ (pg hr/ml) | | |
| | Range A | Range B | Range C | Range A | Range B | Range C |
| baseline- | 2000-4000 | 3593-29942 | 14971.1 ± 10% | 1500-2000 | 1805-15040 | 7519.8 ± 10% |
| adjusted | | | | | | |
| conjugated | | | | | | |
| estrone | | | | | | |
| baseline- | 100-2000 | 210-1751 | 875.5 ± 10% | 80-1000 | 106-882 | 440.6 ± 10% |
| adjusted | | | | | | |
| estrone | | | | | | |
| baseline- | 20-500 | 44-369 | 184.4 ± 10% | 10-250 | 19-162 | 80.8 ± 10% |
| adjusted | | | | | | |
| 17β-estradiol | | | | | | |
| conjugated | 1000-15000 | 1373-11448 | 5723.5 ± 10% | 500-10000 | 760-6336 | 3168.2 ± 10% |
| equilin | | | | | | |
| equilin | 30-500 | 43-361 | 180.3 ± 10% | 15-250 | 23-195 | 97.3 ± 10% |

⁽¹⁾range determined after administration of the seventh dose of the at least daily for 7 consecutive days regimen.
⁽²⁾range determined after administration of the thirteenth dose of the twice per week for at least two weeks regimen.
All values are average (mean) values.

In some embodiments, the method of the present invention provides to the female 48 hours after the seventh dose during the (a) 7 consecutive days of administration a baseline-adjusted estrone AUC_{0-24 hrs} of 210 pg hr/ml to 1751 pg hr/ml. In some embodiments, the method of the present invention can provide to the female 48 hours after the seventh dose during the (a) 7 consecutive days of administration an equilin AUC_{0-24 hrs} of 43 pg hr/mL to 361 pg hr/mL. In some embodiments, the method of the present invention can provide to the female 48 hours after the seventh dose during the (a) 7 consecutive days of administration a baseline-adjusted 17β-estradiol AUC_{0-24 hrs} of 44 pg hr/mL to 369 pg hr/mL.

In some embodiments, the method of the present invention provides to the female 48 hours after the thirteenth dose during the (b) twice per week for at least two weeks of administration (b) a baseline-adjusted estrone AUC_{0-24 hrs} of 106 pg hr/mL to 882 pg hr/mL. In some embodiments, the method of the present invention provides to the female 48 hours after the thirteenth dose during the (b) twice per week for at least two weeks of administration an equilin AUC_{0-24 hrs} 23 pg hr/mL to 195 pg hr/mL. In some embodiments, the method of the present invention provides to the female 48 hours after the thirteenth dose during the (b) twice per week for at least two weeks of administration a baseline-adjusted 17β-estradiol AUC_{0-24 hrs} of 19 pg hr/mL to 162 pg hr/mL.

As described herein, the peak, or maximum plasma concentration (Cₘₐₓ) of estrogens can vary. Suitable Cₘₐₓ of the various estrogens are described in Table 5.

TABLE 5

| | At least daily for 7 consecutive | | | Twice per week for at least 2 | | |
|---|---|---|---|---|---|---|
| | days regimen⁽¹⁾ (pg/ml) | | | weeks regimen⁽²⁾ (pg/ml) | | |
| | Range A | Range B | Range C | Range A | Range B | Range C |
| Baseline- | 100-2500 | 231-1926 | 963.4 ± 10% | 100-1500 | 145-1210 | 604.5 ± 10% |
| adjusted | | | | | | |
| conjugated | | | | | | |
| estrone | | | | | | |
| baseline- | 5-200 | 15-124 | 61.7 ± 10% | 20-100 | 9-71 | 35.7 ± 10% |
| adjusted | | | | | | |
| estrone | | | | | | |
| baseline- | 1-50 | 3-25 | 12.7 ± 10% | 0-25 | 1-14 | 7.0 ± 10% |
| adjusted | | | | | | |
| 17β-estradiol | | | | | | |
| conjugated | 20-1000 | 88-734 | 366.8 ± 10% | 20-700 | 61-510 | 255.2 ± 10% |
| equilin | | | | | | |
| equilin | 1-50 | 3-29 | 14.3 ± 10% | 1-25 | 2-19 | 9.6 ± 10% |

⁽¹⁾range determined after administration of the seventh dose of the at least daily for 7 consecutive days regimen.
⁽²⁾range determined after administration of the thirteenth dose of the twice per week for at least two weeks regimen.
All values are average (mean) values.

In some embodiments, the maximum plasma concentration (Cₘₐₓ) of baseline-adjusted estrone 48 hours after the seventh dose during the (a) 7 consecutive days of administration is 15 pg/mL to 124 pg/mL. In some embodiments, Cₘₐₓ of equilin 48 hours after the seventh dose during the (a) 7 consecutive days of administration is 3 pg/mL to 29 pg/mL. In some embodiments, Cₘₐₓ of baseline-adjusted 17β-estradiol 48 hours after the seventh dose during the (a) 7 consecutive days of administration is 3 pg/mL to 25 pg/mL.

In some embodiments, Cₘₐₓ of baseline-adjusted estrone 48 hours after the thirteenth dose during the (b) twice per week for at least two weeks of administration is 9 pg/mL to 71 pg/mL. In some embodiments, Cₘₐₓ of equilin 48 hours after the thirteenth dose during the (b) twice per week for at least two weeks of administration is 2 pg/mL to 19 pg/mL. In some embodiments, Cₘₐₓ of baseline-adjusted 17β-estradiol 48 hours after the thirteenth dose during the (b) twice per week for at least two weeks of administration is 1 pg/mL to 14 pg/mL.

The time after the vaginal administration of the composition comprising a conjugated estrogen until Cₘₐₓ is achieved can vary. In some embodiments, a time to reach Cₘₐₓ after the vaginal administration is 2 hours to 6 hours.

3. Kits, Dosage Forms and Applicators

The present invention is directed to a kit for administering the vaginal compositions comprising a conjugated estrogen. In some embodiments, the present invention is directed to a kit comprising a dosage form comprising a pharmaceutical vaginal cream composition of the present invention. As used herein, the term “dosage form” refers to a dosage of a composition of the present invention which is administered to a patient in about a 24 hour period. In some embodiments, multiple dosage forms can be contained in one container. In some embodiments, one or more dosage forms are separately packaged from other dosage forms, e.g., individual dosage forms. In some embodiments, the dosage form is a capsule containing the vaginal cream composition comprising a composition of the present invention. In some embodiments, the dosage form comprises a membrane or envelope that surrounds the vaginal cream composition. In some embodiments, the dosage form is a vaginal sachet containing the vaginal cream composition. In some embodiments, the dosage form comprises an amount of the vaginal cream composition contained in a vaginal applicator.

Various numbers of dosage forms can be contained in a single kit. In some embodiments, the kit can comprise from 1 to 60, or 1 to 30, dosage forms comprising a vaginal cream composition of the present invention. In some embodiments, the kit comprises at least 2 dosage forms comprising a vaginal cream composition of the present invention. In some embodiments, the kit comprises at least 7 dosage forms comprising a vaginal cream composition of the present invention. For example, the kit can comprise dosage forms sufficient for initial therapy (e.g., one dose per day for 7 days). In some embodiments, the kit comprises at least 13 dosage forms comprising a composition of the present invention. In some embodiments, the kit can comprise 13 to 14 dosage forms comprising a vaginal cream composition. For example, the kit can comprise dosage forms sufficient for initial therapy plus the remainder of the month (e.g., one dose per day for 7 days plus one dose per day, 2 days per week for the remainder of the month (6-7 doses)). In some embodiments, the kit can comprise at least 8 dosage forms comprising a vaginal cream composition of the present invention. In some embodiments, the kit can comprise 8 to 9 dosage forms comprising a vaginal cream composition of the present invention. For example, the kit can comprise dosage forms sufficient for administration 2 days per week for a month. One of skill in the art could produce additional kits which provide a suitable number of dosage forms within the scope of the invention. For example, a kit comprising dosage forms sufficient for two to six months.

In some embodiments, the present invention is directed to a kit comprising vaginal applicators, wherein each of the vaginal applicators comprises a pharmaceutical vaginal cream composition of the present invention. In some embodiments, the vaginal applicators are disposable. The term “disposable” refers to applicators that are intended to be used once, and then discarded. The disposable applicators can come in any shape and size suitable for applying the vaginal cream of the present invention into a vaginal tract. For example, in some embodiments, the applicator is a syringe. In some embodiments, the applicator is a squeezable tube shaped to allow administration of the vaginal cream directly to the vaginal tract. Alternatively, the kit can comprise a single container comprising multiple doses of the composition. For example, the kit can be a tube containing a month's supply of vaginal cream. In some embodiments, the kit can comprise one or more applicators to apply the vaginal cream composition once it is removed from the container comprising multiple doses. In some embodiments, the kit can comprise one or more devices used to measure an appropriate amount of the composition of the present invention.

The applicator can comprise various amounts of conjugated estrogens. In some embodiments, the applicator comprises a single dosage form or multiple dosage forms of the composition. For example, in some embodiments, the applicator comprises a conjugated estrogen equivalent to 153 μg to 1.55 mg of sodium estrone sulfate. In some embodiments, the applicator comprises a conjugated estrogen equivalent to 60 μg to 775 μg of sodium equilin sulfate. In some embodiments, the applicator comprises a conjugated estrogen equivalent to 1.5 μg to 50 μg of sodium 17β-estradiol sulfate.

The kit of the present invention can contain various amounts of vaginal applicators. In some embodiments, the vaginal applicators are disposable. In some embodiments, the kit comprises 1 to 30 disposable vaginal applicators. In some embodiments, the kit comprises 5 to 20 vaginal applicators. In some embodiments, the kit comprises at least 2 applicators. In some embodiments, the kit comprises at least 7 applicators. In some embodiments, the kit comprises at least 8 applicators. In some embodiments, the kit comprises 8 to 9 vaginal applicators. In some embodiments, the kit comprises at least 13 applicators. In some embodiments, the kit comprises 13 to 14 vaginal applicators. In some embodiments, the vaginal applicators in a kit comprise a conjugated estrogen. In some embodiments, the vaginal applicators in a kit comprise a conjugated estrogen and a stabilizer.

The kit can include one or more containers filled with one or more of the ingredients of the vaginal cream compositions of the invention. In some embodiments, the vaginal cream composition of the present invention is stored in a container essentially impermeable to oxygen. In some embodiments, the vaginal cream composition is purged with an inert gas, e.g., nitrogen gas.

Optionally associated with such container(s) can be a notice or printed instructions. For example, such printed instructions can be in a form prescribed by a governmental agency regulating the manufacture, use or sale of pharmaceuticals or biological products, which notice reflects approval by the agency of the manufacture, use or sale for human administration to treat a menopausal condition. In some embodiments, the kit further comprises printed matter, which, e.g., provides information on the use of the vaginal cream composition to treat a menopausal condition or a pre-recorded media device which, e.g., provides information on the use of the vaginal cream composition to treat a menopausal condition, or a planner.

“Printed matter” can be, for example, one of a book, booklet, brochure or leaflet. The printed matter can describe the use of the vaginal cream composition of the present invention for the treatment of a menopausal condition. Possible formats included, but are not limited to, a bullet point list, a list of frequently asked questions (FAQ) or a chart. Additionally, the information to be imparted can be illustrated in non-textual terms using pictures, graphics or other symbols.

“Pre-recorded media device” can be, for example, a visual media device, such as a videotape cassette, a DVD (digital video disk), filmstrip, 35 mm movie or any other visual media device. Alternately, pre-recorded media device can be an interactive software application, such as a CD-ROM (compact disk-read only memory) or floppy disk. Alternately, pre-recorded media device can be, for example, an audio media device, such as a record, audiocassette or audio compact disk. The information contained on the pre-recorded media device can describe the use of the vaginal cream composition of the present invention for the treatment of a menopausal condition.

A “planner” can be, for example, a weekly, a monthly, a multi-monthly, a yearly, or a multi-yearly planner. The planner can be used as a diary to monitor dosage amounts, to keep track of dosages administered, or to prepare for future events wherein taking a regularly administered vaginal cream composition of the present invention may be difficult. Alternately, the planner can be a calendar which will provide a means to monitor when a dosage has been taken and when it has not been taken. This type of planner will be particularly useful for patients having unusual schedules for administering medication to themselves. Additionally, the planner can be useful for the elderly, or other patient group who may administer medication to themselves and may become forgetful. One skilled in the art will appreciate the variety of planning tools that would be appropriate for use with the present invention.

The kit can also include a container for storing the other components of the kit. The container can be, for example, a bag, box, envelope or any other container that would be suitable for use in the present invention. Preferably, the container is large enough to accommodate each component and/or any administrative devices that may be necessary for a vaginal cream composition of the present invention. However, in some cases, it may be desirable to have a smaller container which can be hidden in a patient's pocketbook, briefcase or pocket.

The present invention is also directed to a method of delivery of the composition of the present invention to a patient in need thereof, the method comprising (a) registering in a computer readable medium the identity of a physician permitted to prescribe the vaginal cream composition; (b) providing the patient with counseling information concerning the risks attendant to the vaginal cream composition; (c) obtaining informed consent from the patient to receive the vaginal cream composition despite the attendant risks; (d) registering the patient in a computer readable medium after obtaining their informed consent; and (e) permitting the patient access to the vaginal cream composition.

The drug delivery methods of the present invention involve, inter alia, registering in a computer readable storage medium physicians who are qualified to prescribe the vaginal cream composition of the present invention. Once registered in the computer readable storage medium, the physician can be eligible to prescribe the vaginal cream composition to a patient in need thereof. Generally speaking, in order to become registered in the computer readable storage medium, the physician may be required to comply with various aspects of, for example, providing patient education and counseling. The registration of the physician in the computer readable storage medium can be achieved by providing the physician, for example, by mail, facsimile transmission, or on-line transmission, with a registration card or form, preferably together with educational materials concerning the vaginal cream composition of the present invention. The physician can complete the registration card or form by providing information requested therein, and the registration card or form can be returned to the manufacturer or distributor of the vaginal cream composition of the present invention, or other authorized recipient of the registration materials, for example, by mail, facsimile transmission or on-line transmission. The physician's information in the registration card or form is then entered into the computer readable storage medium. Suitable computer readable storage media which can be employed for registration of the physicians (as well as patients, as discussed below) will be apparent to one of ordinary skill in the art, once in possession of the teaching of the present application.

In the course of examination of a patient, including a patient suffering from a menopausal condition, the physician may determine that the patient's condition can be improved by the administration of the vaginal cream composition of the present invention. Prior to prescribing the vaginal cream composition of the present invention, the physician can counsel the patient, for example, on the various risks and benefits associated with the vaginal cream composition. The patient can be provided full disclosure of all the known and suspected risks associated with the vaginal cream composition. Such counseling can be provided verbally, as well as in written form. In some embodiments, the physician can provide the patient with literature materials on the vaginal cream composition, such as product information, educational materials, and the like.

In addition to receiving counseling on the risks attendant to the vaginal cream composition of the present invention, the methods of the invention further require the patient to fill out an informed consent form which is signed by the patient. Upon the completion of the informed consent form, the patient can be registered in a computer readable storage medium. The computer readable storage medium in which the patient is registered can be the same as, or different from, the computer readable storage medium in which the physician is registered.

The registration into one or more computer readable storage media of the physician and patient, according to the methods describe herein, provides a means to monitor and authorize access to the vaginal cream composition of the present invention. Thus, the computer readable storage medium can serve to deny access to patients who fail to abide by the methods of the present invention. In some embodiments, access to the vaginal cream composition of the invention is in the form of a prescription, wherein the prescribing physician is registered in a computer readable storage medium, has provided counseling to the patient concerning the attendant risks of the vaginal cream composition, and has obtained informed consent from the patient, prior to prescribing the vaginal cream composition to the patient in need thereof.

The present invention is also directed to methods of educating consumers about the use of a vaginal cream composition of the invention, the method comprising distributing the vaginal cream composition with consumer information at a point of sale. In some embodiments, the distribution will occur at a point of sale having a pharmacist or healthcare provider.

As used herein, the term “consumer information” can include, but is not limited to, an English language text, non-English language text, visual image, chart, telephone recording, website, and access to a live costumer service representative. In some embodiments of the present invention, consumer information will provide directions for use of the vaginal cream composition of the present invention, appropriate age use, indication, contraindications, appropriate dosing, warnings, telephone number of website address. In some embodiments, the method further comprises providing professional information to relevant persons in a position to answer consumer questions regarding the vaginal cream composition.

As used herein, the term “professional information” includes, but is not limited to, information concerning the vaginal cream composition of the present invention designed to enable a healthcare professional to answer costumer questions regarding the vaginal cream composition.

A “relevant person,” as used herein, includes, for example, a physician, physician assistant, nurse practitioner, pharmacist and customer service representative.

All of the various embodiments or options described herein can be combined in any and all variations.

The following examples are further illustrative of the present invention, but are not to be construed to limit the scope of the present invention.

### EXAMPLE 1

Vaginal cream formulations were prepared as described in Table 6. Formulation A contained the stabilizers edetate disodium, butylated hydroxyanisole (BHA) and butylated hydroxytoluene (BHT); Formulation B contained no stabilizers as defined herein.

TABLE 6

| | Formulation A | | Formulation B |
|---|---|---|---|
| Ingredients | (mg) | | (mg) |
| Synthetic conjugated estrogens* | 0.625 | | 0.625 |
| Water (purified) | 472.75 | | 475.0 |
| Water (added to Sodium Hydroxide) | 10.0 | | 10.0 |
| Sodium Hydroxide | 0.25 | | 0.25 |
| Glycerin | 186.375 | | 186.375 |
| Sodium Lauryl Sulfate | 3.0 | | 3.0 |
| Benzyl Alcohol, NF | 10.0 | | 10.0 |
| Sodium Phosphate Dibasic, | 1.50 | | 0.9 |
| Anhydrous, NF | | | |
| Edetate Disodium, USP | 0.5 | | 0 |
| Cetyl Esters Wax, NF | 90.0 | | 90.0 |
| Light Mineral Oil, NF | 68.25 | | 70.0 |
| Propylene Glycol Monostearate | 70.0 | | 70.0 |
| Cetyl Alcohol, NF | 50.0 | | 50.0 |
| Methyl Stearate | 15.0 | | 15.0 |
| Beeswax, NF | 10.0 | | 10.0 |
| Glyceryl Monostearate | 10.0 | | 10.0 |
| Butylated Hydroxyanisole, Food Grade | 1.25 | | 0 |
| Butylated Hydroxytoluene, Food Grade | 0.50 | | 0 |
| Total Weight | 1000 | mg | 1001.5 |

*Conjugated estrogens in glycerin base.

The synthetic conjugated estrogens in Example 1 contained a mixture of 9 estrogens. The relative amounts of the conjugated estrogens (CE) are presented in Example 2.

### EXAMPLE 2

Quantitative analysis was performed on the conjugated estrogens used for Formulation B. Relative amounts of each estrogen are presented in Table 7. The analysis method conforms to USP 23, supplement 2. The results are found in Table 7.

TABLE 7

CE used for Formulation B

| Estrogen | Analysis #1^{a} | Analysis #2^{b} |
|---|---|---|
| 17α-estradiol | 5.1 | 3.5-7.0 |
| 17α-dihydroequilin | 16.2 | 14.0-18.0 |
| 17β-dihydroequilin | 1.8 | 1.5-3.5 |
| Estrone | 55.8 | 55-61 |
| Equilin | 27.9 | 24-31 |
| 17β-estradiol | 1.1 | 0.5-2.0 |
| 17α-dihydroequilenin | 0.5 | 0.1-2.75 |
| 17β-dihydroequilenin | 0.5 | 0.1-0.76 |
| Equilenin | 0.9 | 0.5-3.25 |

^{a}% of total CE in glycerin (from a 37.5 mg estrogen/g (dry weight basis) formulation).
^{b}% of total CE in solution (from a 125 g estrogen/L formulation). All estrogens reported as the sodium salts of the 3-monophosphate esters.

### EXAMPLE 3

Stability of pharmaceutical vaginal cream compositions was investigated. Two separate preparations were prepared. As described in Example 1, Formulation A contained the stabilizers BHA and BHT. Formulation B was identical to Formulation A, except it did not contain a stabilizer. Aliquots of Formulation A and Formulation B were either stored (a) under normal conditions of 25° C. at 60% relative humidity (RH) for 1 month, or (b) under accelerated conditions of 40° C. at 75% RH for 2 weeks or 1 month, as exemplified in Table 8. At the designated time, the compositions were assayed by HPLC to determine degradation products. The values in Table 8 represent the percent by weight of the specified estrogen compared to the total estrogen amount. The estrogens designated with an asterisk (*) indicate estrogenic degradation products.

TABLE 8

| | | | | |
|---|---|---|---|---|
| | | 1 month | 2 weeks | 1 month |
| Estrogen | Initial | 25° C./60% RH | 40° C./75% RH | 40° C./75% RH |

Formulation B

| | | | | |
|---|---|---|---|---|
| Estrone | 62.62 | 62.15 | 62.68 | 62.66 |
| Equilin | 29.85 | 29.62 | 29.68 | 29.27 |
| 17α-dihydroequilin | 17.58 | 17.42 | 17.41 | 17.14 |
| 17β-dihydroequilin | 1.88 | 1.90 | 1.83 | 1.85 |
| 17β-estradiol | 0.99 | 1.01 | 1.03 | 1.04 |
| 17α-estradiol | 5.84 | 5.86 | 5.87 | 5.73 |
| 17β-dihydroequilenin* | 0.51 | 0.46 | 0.50 | 0.52 |
| 17α-dihydroequilenin* | 2.48 | 2.77 | 3.19 | 3.85 |
| Equilenin* | 1.61 | 1.68 | 1.98 | 2.23 |

Formulation A

| | | | | |
|---|---|---|---|---|
| Estrone | 57.61 | 57.88 | 57.84 | 57.86 |
| Equilin | 28.00 | 28.05 | 27.65 | 27.17 |
| 17α-dihydroequilin | 16.57 | 16.57 | 16.22 | 15.91 |
| 17β-dihydroequilin | 1.84 | 1.77 | 1.75 | 1.68 |
| 17β-estradiol | 0.96 | 0.91 | 0.94 | 1.03 |
| 17α-estradiol | 5.50 | 5.56 | 5.35 | 5.39 |
| 17β-dihydroequilenin* | 0.40 | 0.42 | 0.41 | 0.49 |
| 17α-dihydroequilenin* | 1.79 | 2.00 | 2.43 | 3.30 |
| Equilenin* | 1.21 | 1.23 | 1.48 | 1.92 |

### EXAMPLE 4

Multiple dose pharmacokinetics of estrogens from Formulation B of Example 1 were assessed in a Phase I open-label study. Three treatments were employed: Treatment A with a vaginal application of 1.250 mg of conjugated estrogens daily for a total of 21 days; Treatment B with 0.625 mg daily for a total of 21 days; and Treatment C with 1.250 mg conjugated estrogens daily for 7 days followed by 1.250 mg twice weekly for a total of 26 days. Thirty healthy postmenopausal females were enrolled in the study and assigned to one of the three treatment groups. Twenty-nine subjects completed all components of the study. Plasma samples for pharmacokinetic profile determination were obtained after dosing on Day 1 and Day 21 for Treatments A and B; samples were obtained after dosing on Day 1, Day 7, and Day 26 for Treatment C. Plasma samples were assayed for estradiol, free and total estrone, and free and total equilin. From the assay results, plasma levels of conjugated 17β-estradiol and conjugated estrone, and subsequently plasma levels of baseline-adjusted (BA) 17β-estradiol and estrone were also calculated.

The mean pharmacokinetic profiles for estrone, 17β-estradiol, and equilin are presented in FIGS. 1-5. For the majority of the analytes, the exposure from the vaginal cream application comprising estrogens was less after multiple dosing compared to the exposure after a single dose, both in the extent parameter (AUC_{0-24 hrs}) and in the rate parameter (Cₘₐₓ). Thus, in general, the exposure to exogenous estrogens decreased after multiple applications of Formulation B vaginal cream.

Upon multiple dosing by oral route, the systemic exposures (AUC_{0-24 hrs}) to estrogenic components increase until steady state levels are reached (Stevens et al., J. Clin. Pharmacol., 42:332-341 (2002)). Upon multiple dosing by vaginal cream application, the systemic exposures remain unchanged or decrease until steady state is reached. This is demonstrated in Table 9.

TABLE 9

| | | | |
|---|---|---|---|
| AUC_{0-24 hr} | AUC_{0-24 hr} | Cₘₐₓ | Cₘₐₓ |
| Day 1 | SS | Day 1 | SS |
| (pg hr/mL) | (pg hr/mL) | (pg hr/mL) | (pg hr/mL) |

Oral Administration (Stevens et al., 2002)

| | | | | |
|---|---|---|---|---|
| BA 17β- | 98.6 | 200.0 | 6.8 | 11.6 |
| Estradiol | | | | |
| BA Estrone | 605.4 | 1197.0 | 40.3 | 77.6 |
| Equilin | 294.0 | 498.0 | 22.2 | 37.0 |

Treatment A

| | | | | |
|---|---|---|---|---|
| BA 17β- | 137.4 | 154.3 | 13.4 | 10.5 |
| Estradiol | | | | |
| BA Estrone | 722.3 | 648.2 | 75.3 | 49.6 |
| Equilin | 168.4 | 133.2 | 19.2 | 11.6 |

Treatment B

| | | | | |
|---|---|---|---|---|
| BA 17β- | 97.3 | 99.2 | 10.9 | 7.4 |
| Estradiol | | | | |
| BA Estrone | 492.7 | 412.4 | 57.8 | 29.4 |
| Equilin | 113.2 | 86.1 | 15.0 | 7.7 |

Treatment C

| | | | | |
|---|---|---|---|---|
| BA 17β- | 172.2 | 38.3* | 18.4 | 7.0 |
| Estradiol | | | | |
| BA Estrone | 964.1 | 218.3* | 112.1 | 35.7 |
| Equilin | 249.8 | 32.1* | 33.0 | 9.6 |

*Average 24 hour exposure estimated from 7-day exposure.
BA = baseline-adjusted values.

For an equivalent daily dose, the steady state systemic exposure from the cream is considerably less than the exposure from oral administration. The steady state cream (Treatment C)/steady state oral tablet pharmacokinetic parameter AUC_{0-24 hrs} ratios are as follows: 0.19 for BA 17β-estradiol, 0.18 for BA estrone, and 0.06 for Equilin. Thus, compared to the estimated pharmacokinetic parameter values for a 0.625 mg daily oral dose (weekly total dose: 4.375 mg), a twice-weekly dose of 1.250 mg Formulation B cream (weekly total dose: 2.50 mg) resulted in daily exposures that are lower by 81% for baseline-adjusted 17β-estradiol, by 82% for baseline-adjusted estrone, and by 94% for equilin.

### EXAMPLE 5

A randomized, double-blind, placebo-controlled, multicenter study was conducted to compare the safety and efficacy of the Formulation B vaginal cream. A total of 278 patients were randomized (57 to Formulation B vaginal cream daily, 57 to Formulation B vaginal cream twice weekly, 56 to daily placebo, 52 to twice-weekly placebo, and 56 to Premarin® vaginal cream) for a total treatment period of 12 weeks. The maturation index, vaginal pH and change in severity of the most bothersome self-assessed symptom were evaluated over the 12 week treatment period.

There were seven clinic visits during the trial: one screening visit (Visit 1, Week −4), one randomization visit (Visit 2, Week 0), and five treatment visits (Visits 3, 4, 5, 5a, and 6, at Study Weeks 2, 3, 4, 8, and 12 respectively).

At Visit 1, potential patients signed an informed consent and provided medical history information. Tests and procedures performed at the screening visit included measurements of body weight, height, and vital signs (blood pressure and pulse); physical examination (including breast and pelvic examinations); a mammogram (unless documented results of a previous NORMAL mammogram within 36 weeks were available); transvaginal ultrasound (TVU) (to confirm hysterectomy if surgical records were not available) and an endometrial biopsy for patients with an intact uterus; Papanicolaou (Pap) smear; urine pregnancy test (for women with an intact uterus); serum chemistry, hematology, and urinalysis panels; serum triglycerides; serum follicle stimulating hormone (FSH); serum estradiol; vaginal cytology and pH; and sCTX levels. An Investigator's Assessment of vaginal atrophy was completed with categories including vaginal atrophy, color of the vaginal epithelium, and dryness; vaginal tissue integrity/friability; and vaginal tissue petechiae. Patients completed a written self-assessment of vaginal atrophy that included answering questions regarding vaginal dryness and itching, and problems with urination, libido, and intercourse. Patients also indicated which of the symptoms was most bothersome. Eligible patients had to have at least one of the symptoms of vaginal atrophy rated as moderate or severe that required treatment in order to qualify for the study. The time period to complete all screening procedures was up to 4 weeks (28 days) between Visit 1 and Visit 2.

Eligible patients returned to the clinic for randomization to one of five treatment groups. Patients were randomized to receive Formulation B vaginal cream daily or twice weekly, Premarin® vaginal cream daily, or Placebo vaginal cream daily or twice weekly for the 12-week treatment period. Specifically, patients in the daily dosing groups applied 2 grams of cream once daily for 3 weeks, withheld applications for 1 week, and then repeated cyclically (3 weeks on, 1 week off) for the remainder of the 12-week treatment period. Patients assigned to the twice weekly dosing groups applied 2 grams of cream daily for 1 week, then applied it twice weekly (Tuesdays and Fridays) for the remainder of the 12-week treatment period. Patients were instructed to apply the cream at the same time in the morning or evening, except on clinic visit days when they were to wait until after vaginal smear samples had been collected. Testing and procedures at the baseline/randomization visit included measurements of body weight and vital signs (blood pressure and pulse); serum estradiol; and sCTX levels.

During the 12-week treatment period, patients returned to the clinic at Weeks 2, 3, 4, 8, and 12. At each clinic visit during the treatment period, body weight and vital signs (blood pressure and heart rate) were assessed. Patients completed the self-assessment of vaginal atrophy symptoms, including answering a question regarding which symptom was most bothersome. The investigator completed a similar assessment at each treatment clinic visit regarding the signs of vaginal atrophy. At Weeks 2, 3, 4, and 12, samples were collected for vaginal cytology and pH measurements. At Weeks 4 and 12, sCTX levels were measured. At the end of treatment (Week 12), final measurements of serum estradiol and FSH were completed. In addition, at the final clinic visit serum chemistry, hematology, and urinalysis testing were completed, and a physical examination including breast and pelvic examinations was performed. An endometrial biopsy was done on all patients with a uterus.

Primary Efficacy Variables

The primary measures of efficacy were developed based on discussion with the FDA as the change from baseline to end of treatment for the three co-primary response measurements: (1) maturation index; (2) vaginal pH; and (3) severity of the patient's most bothersome symptom.

1. Maturation Index

The mean change in the maturation index of the vaginal mucosa values between baseline (Week −4) and end of treatment (Week 12) was calculated by counting the number of parabasal, intermediate and superficial cells and calculating the percentage of each cell type. Unless otherwise specified, the use of the term “Baseline” will refer to Week −4 and “End of Treatment” will refer to Week 12 (or the patient's last visit). The percentages were then used in the following equation to determine the maturation index.
Maturation Index=(% Parabasal cells×0)+(% Intermediate cells×0.5)+(% Superficial cells×1.0)

Patients in the active treatment groups (Formulation B Daily, Formulation B Twice Weekly, and Premarin® Daily) exhibited significantly greater mean increases in the maturation index between baseline and end of treatment over their corresponding placebo control groups (Table 10).

TABLE 10

| | | Base- | Mean | | Prob > |
|---|---|---|---|---|---|
| Treatments | N | line | Change | Difference | F* |
| Formulation B Daily | 48 | 38.13 | 34.34 | 26.74 | <0.0001 |
| Placebo Daily | 49 | 40.27 | 7.60 | | |
| Formulation B 2 × Weekly | 46 | 38.76 | 32.53 | 29.70 | <0.0001 |
| Placebo 2 × Weekly | 45 | 39.24 | 2.83 | | |
| Premarin ® Daily | 45 | 39.06 | 34.36 | 26.76 | <0.0001 |
| Placebo Daily | 49 | 40.27 | 7.60 | | |
| Formulation B Daily** | 48 | 38.13 | 34.34 | 1.81 | 0.6816 |
| Formulation B 2 × Weekly | 46 | 38.76 | 32.53 | | |
| Formulation B Daily** | 48 | 38.13 | 34.34 | −0.02 | 0.6336 |
| Premarin ® Daily | 45 | 39.06 | 34.36 | | |
| Formulation B 2 × Weekly** | 46 | 38.76 | 32.53 | −1.83 | 0.9468 |
| Premarin ® Daily | 45 | 39.06 | 34.36 | | |

*Prob > F = Test for significant difference between treatment.
**Test between active treatment groups was determined by the results between active and placebo.

The mean maturation index at baseline was similar for the five treatment groups (38.1, 38.8, 39.1, 40.3, and 39.2 for the Formulation B Daily, Formulation B Twice Weekly, Premarin® Daily, Placebo Daily, and Placebo Twice Weekly groups, respectively). At end of treatment the mean increase from baseline in the maturation index was 34.3, 32.5, 34.4, 7.6, and 2.8 for each treatment group, respectively. This represents a highly significant difference between the active treatment groups and their corresponding placebo controls (p<0.0001). The largest differential effect versus placebo was observed for Formulation B Twice Weekly (29.7) followed by Premarin® Daily (26.8) and Formulation B Daily (26.7). There was no detectable difference between the active treatment groups. However, the power of a statistical test comparing any pair of active treatment arms is insufficient to allow for any conclusions regarding their comparability

2. Vaginal pH

The mean change in vaginal pH between baseline and end of treatment was calculated by measuring the vaginal pH by inserting a standardized pH paper into the vagina and comparing the results to the manufacturer's color chart.

Patients in the active treatment groups (Formulation B Daily, Formulation B Twice Weekly, and Premarin® Daily) exhibited significantly greater mean reductions in vaginal pH between baseline and end of treatment over their corresponding placebo control groups (Table 11).

TABLE 11

| | | Base- | Mean | | Prob > |
|---|---|---|---|---|---|
| Treatments | N | line | Change | Difference | F* |
| Formulation B Daily | 48 | 5.53 | −0.76 | −0.49 | <0.0001 |
| Placebo Daily | 49 | 5.88 | −0.27 | | |
| Formulation B 2 × Weekly | 46 | 5.60 | −0.98 | −1.12 | <0.0001 |
| Placebo 2 × Weekly | 45 | 5.47 | 0.14 | | |
| Premarin ® Daily | 45 | 5.55 | −0.77 | −0.50 | <0.0001 |
| Placebo Daily | 49 | 5.88 | −0.27 | | |
| Formulation B Daily** | 48 | 5.53 | −0.76 | 0.22 | 0.1784 |
| Formulation B 2 × Weekly | 46 | 5.60 | −0.98 | | |
| Formulation B Daily** | 48 | 5.53 | −0.76 | 0.01 | 0.9848 |
| Premarin ® Daily | 45 | 5.55 | −0.77 | | |
| Formulation B 2 × Weekly** | 46 | 5.60 | −0.98 | −0.21 | 0.1903 |
| Premarin ® Daily | 45 | 5.55 | −0.77 | | |

*Prob > F = Test for significant difference between treatment.
**Test between active treatment groups was determined by the results between active and placebo.

The mean vaginal pH at baseline was similar for the five treatment groups (5.5, 5.6, 5.6, 5.9, and 5.5 for the Formulation B Daily, Formulation B Twice Weekly, Premarin® Daily, Placebo Daily, and Placebo Twice Weekly groups, respectively). At end of treatment, the mean change from baseline in the vaginal pH was −0.76, −0.98, −0.77, −0.27 and +0.14 for each treatment group, respectively. This results in a difference from placebo of −1.12, −0.50, and −0.49 for the Formulation B Twice Weekly, Premarin® Daily, and Formulation B Daily treatment groups, respectively, all of which are highly significant (p<0.0001). There was no detectable difference between the active treatment groups.

3. Most Bothersome Symptoms

The mean change in the severity of the symptom identified by the patient at baseline as being the most bothersome, between baseline and the end of treatment. This Patient Self-Assessment consisted of 10 questions about the severity of symptoms graded on a scale of 0 to 3 (None, Mild, Moderate, or Severe), and additional questions about the patient's change in libido and irritation caused by the cream. The most bothersome was the symptom identified by the patient on the baseline Patient Self-Assessment from one of the following seven symptoms: (1) vaginal dryness; (2) vaginal irritation/itching; (3) vaginal soreness; (4) difficulty passing urine; (5) frequent urination; (6) pain during intercourse; and (7) bleeding after intercourse.

Table 12 summarizes the mean severity at baseline and end of treatment, and the change between these two time points for the most bothersome symptom. The most bothersome symptom was identified by the patient from a list of the seven different symptoms of vaginal atrophy included on the Patient Self-Assessment at the baseline visit. At baseline, the breakdown of symptoms chosen as most bothersome was as follows: 123 (52.8%) vaginal dryness, 25 (10.7%) vaginal irritation or itching, 17 (7.3%) vaginal soreness, 6 (2.6%) difficulty passing urine, 33 (14.2%) frequent urination, 28 (12.0%) pain during intercourse, and 1 (0.4%) bleeding after intercourse. The severity of the symptom identified as most bothersome at baseline for each patient was then averaged across patients within each treatment group. A similar calculation was performed at the end of treatment for the same symptom considered most bothersome at baseline, whether or not that symptom was still considered the patient's most bothersome symptom. Therefore, built into this measure is not only the variability associated with how different patients experience their symptoms, but also the variability associated with the symptom that was chosen as most bothersome. In addition, this way of defining change in severity of symptoms makes it difficult to pre-specify a clinically meaningful change that would constitute clear evidence of an efficacious response that is statistically significantly superior to placebo.

Results indicated a consistently greater mean reduction in the severity of the most bothersome symptom for each active treatment group compared with its placebo (Table 12). Slightly greater reductions are seen for the daily active dosing regimens than for the twice-weekly regimen; no discernable difference was observed between the two placebo dosing regimens.

TABLE 12

| | | | Mean | | |
|---|---|---|---|---|---|
| Treatments | N | Baseline | Change | Difference | Prob > F* |
| Formulation B Daily | 48 | 2.40 | −1.27 | −0.15 | 0.3984 |
| Placebo Daily | 49 | 2.47 | −1.12 | | |
| Formulation B 2 × | 46 | 2.37 | −1.22 | −0.15 | 0.2139 |
| Weekly | | | | | |
| Placebo 2 × Weekly | 45 | 2.53 | −1.07 | | |
| Premarin ® Daily | 45 | 2.29 | −1.38 | −0.26 | 0.1389 |
| Placebo Daily | 49 | 2.47 | −1.12 | | |

*Prob > F = Test for significant difference between treatment.

4. Efficacy Conclusions

Formulation B vaginal cream administered daily and twice weekly were both shown to be safe and effective in the treatment of vulvovaginal atrophy in postmenopausal women. Significant increases in the maturation index and significant decreases in vaginal pH were observed following up to 12 weeks of treatment. Reduction in the severity of the most bothersome symptom reported on the Patient's Self-Assessment of vaginal atrophy was also observed. The magnitude of treatment effect for all three of these endpoints was similar for the daily and twice-weekly regimens. The incidence of adverse events was comparable across the three active treatment and two placebo control groups. In addition, the majority of patients reported no cream irritation (average less than mild).

This example demonstrates that a twice-weekly regimen of Formulation B vaginal cream is associated with a beneficial treatment effect of comparable magnitude to the daily regimens of Formulation B vaginal cream and Premarin® vaginal cream. In the present inventions, the twice-weekly regimen is associated with estrogen exposure that is approximately half of that associated with the daily regimen, while conferring a satisfactory level of efficacy. Formulation B vaginal cream applied twice weekly was an effective treatment for vulvovaginal atrophy and, although not formally compared with either the once-daily Formulation B vaginal cream formulation or to Premarin® Cream, twice weekly Formulation B vaginal cream provided a level of efficacy, safety, and tolerability that was comparable to the daily regimens.

### EXAMPLE 5

Adverse events (AEs) were reported during the patient's regularly scheduled visits to the investigational site. Site personnel recorded the information regarding each event on the AE page of the CRF. Treatment-emergent AEs were reported by 192 (69.3%) of the 277 patients in the Safety cohort. There was no significant difference between the treatment groups with respect to proportion reporting at least one treatment-emergent AE; 63.2%, 63.2%, 81.8%, 71.4%, and 67.3% of the patients in the Formulation B Daily, Formulation B Twice Weekly, Premarin® Daily, Placebo Daily, and Placebo Twice Weekly treatment groups, respectively, reported an adverse event.

Table 13 summarizes the treatment-emergent AEs associated with vascular disorders.

TABLE 13

| | Formulation B | Placebo | Formulation B | Placebo 2 × | Premarin |
|---|---|---|---|---|---|
| | Daily | Daily | 2 × Weekly | Weekly | Daily |
| Hemorrhage | 0 (0.00%) | 0 (0.00%) | 0 (0.00%) | 1 (1.92%) | 0 (0.00%) |
| Hot Flashes | 1 (1.75%) | 6 (10.71%) | 1 (1.75%) | 1 (1.92%) | 1 (1.82%) |
| Hypertension | 0 (0.00%) | 0 (0.00%) | 0 (0.00%) | 0 (0.00%) | 1 (1.82%) |
| aggravated | | | | | |
| Total | 1 (1.75%) | 6 (10.71%) | 1 (1.75%) | 2 (3.85%) | 2 (3.64%) |

The term “treatment-emergent AEs” refers to AEs that occurred on or after the first dose through the date of study completion (including events that occurred after the last treatment, but before the patient completed the study). Adverse events judged to be possibly related or related to study drug by the investigator were considered to be treatment-related AEs. Treatment-emergent AEs did not appear to be dose-related. The distribution of AEs or patients discontinuing due to adverse events appeared to be randomly distributed across the five treatment groups.

A reduction of hot flashes occurred in females taking Formulation B daily compared to those taking the placebo daily. Likewise, there was a low incidence of hot flashes in females taking Formulation B twice weekly. This suggests that twice weekly administration can possibly be involved with systematically effecting menopausal conditions.

These examples illustrate possible embodiments of the present invention. While the invention has been particularly shown and described with reference to some embodiments thereof, it will be understood by those skilled in the art that they have been presented by way of example only, and not limitation, and various changes in form and details can be made therein without departing from the spirit and scope of the invention. Thus, the breadth and scope of the present invention should not be limited by any of the above-described exemplary embodiments, but should be defined only in accordance with the following claims and their equivalents.

All documents cited herein, including journal articles or abstracts, published or corresponding U.S. or foreign patent applications, issued or foreign patents, or any other documents, are each entirely incorporated by reference herein, including all data, tables, figures, and text presented in the cited documents.

1. A pharmaceutical vaginal cream composition comprising:
(a) a conjugated estrogen, and
(b) a stabilizer.

2. The composition of claim 1, wherein said conjugated estrogen comprises two or more conjugated estrogens.

3. The composition of claim 1, wherein said stabilizer is selected from the group consisting of butylated hydroxyanisole, butylated hydroxytoluene, ascorbic acid and its esters, vitamin E and its esters, sodium bisulfite, sodium metabisulfite, 3-dehydroshikimic acid, tocopherols and their esters, alkyl gallates, chelating agents, EDTA, citric acid, benzyl alcohol, and combinations thereof.

4. (canceled)

5. The composition of claim 1, wherein said conjugated estrogen comprises sodium estrone sulfate, sodium equilin sulfate, sodium 17α-dihydroequilin sulfate, sodium 17β-dihydroequilin sulfate, sodium 17α-estradiol sulfate, sodium 17β-estradiol sulfate, sodium equilenin sulfate, sodium 17α-dihydroequilenin sulfate, sodium 17β-dihydroequilenin sulfate or a combination thereof.

6-8. (canceled)

9. The composition of claim 1, further comprising a pharmaceutically acceptable excipient.

10. The composition of claim 9, wherein said pharmaceutically acceptable excipient is selected from the group consisting of a stiffening agent, an oil, a solvent, an emulsifier, a humectant, a buffering agent, and combinations thereof.

11-18. (canceled)

19. The composition of claim 1, wherein said composition is in a dosage form, wherein said dosage form comprises 0.3 mg to 2.5 mg of conjugated estrogens, wherein said conjugated estrogens consist of:
(a) 3.5% to 7.0% by weight sodium 17α-estradiol sulfate;
(b) 10.0% to 19.0% by weight sodium 17α-dihydroequilin sulfate;
(c) 0.5% to 3.5% by weight sodium 17β-dihydroequilin sulfate;
(d) 51.0% to 62.0% by weight sodium estrone sulfate;
(e) 20.0% to 31.0% by weight sodium equilin sulfate;
(f) 0.5% to 2.0% by weight sodium 17β-estradiol sulfate;
(g) 0.2% to 5.0% by weight sodium 17α-dihydroequilenin sulfate;
(h) 0.1% to 1.5% by weight sodium 17β-dihydroequilenin sulfate; and
(i) 0.5% to 6.5% by weight sodium equilenin sulfate;
or an amount of a mixture of conjugated estrogens that provides an estrogenic effect equivalent to that produced by the 0.3 mg to 2.5 mg of conjugated estrogens as defined in (a)-(i).

20. The composition of claim 1, wherein said composition is in a dosage form, wherein said dosage form comprises a conjugated estrogen equivalent to 153 μg to 1.55 mg of sodium estrone sulfate.

21. The composition of claim 1, wherein said composition is in a dosage form, wherein said dosage form comprises a conjugated estrogen equivalent to 60 μg to 775 μg of sodium equilin sulfate.

22. The composition of claim 1, wherein said composition is in a dosage form, wherein said dosage form comprises a conjugated estrogen equivalent to 1.5 μg to 50 μg of sodium 17β-estradiol sulfate.

23. A kit comprising a dosage form comprising the composition of claim 1.

24-30. (canceled)

31. A kit comprising a disposable vaginal applicator and said composition of claim 1.

32-36. (canceled)

37. The kit of claim 31, wherein said composition is contained in said disposable vaginal applicator.

38. The kit of claim 31, further comprising printed instructions for use of said kit.

39. The kit of claim 38, wherein said printed instructions provide a method of using said disposable applicator, wherein said method comprises vaginally administering said composition twice per week for at least 2 weeks.

40. The kit of claim 38, wherein said printed instructions provide a method of using said disposable applicator, wherein said method comprises vaginally administering said composition (a) at least once daily for at least 7 consecutive days, then (b) twice per week for at least 2 weeks.

41. A disposable applicator comprising the composition of claim 1.

42. A method of treating a menopausal condition in a female in need thereof, said method comprising administering the composition of claim 1.

43. The method of claim 42, wherein said composition is in a dosage form, wherein said dosage form comprises 0.3 mg to 2.5 mg of conjugated estrogens, wherein said conjugated estrogens consist of:
(a) 3.5% to 7.0% by weight sodium 17α-estradiol sulfate;
(b) 10.0% to 19.0% by weight sodium 17α-dihydroequilin sulfate;
(c) 0.5% to 3.5% by weight sodium 17β-dihydroequilin sulfate;
(d) 51.0% to 62.0% by weight sodium estrone sulfate;
(e) 20.0% to 31.0% by weight sodium equilin sulfate;
(f) 0.5% to 2.0% by weight sodium 17β-estradiol sulfate;
(g) 0.2% to 5.0% by weight sodium 17α-dihydroequilenin sulfate;
(h) 0.1% to 1.5% by weight sodium 17β-dihydroequilenin sulfate; and
(i) 0.5% to 6.5% by weight sodium equilenin sulfate;
or an amount of a mixture of conjugated estrogens that provides an estrogenic effect equivalent to that produced by the 0.3 mg to 2.5 mg of conjugated estrogens as defined in (a)-(i).

44. The method of claim 42, wherein said composition is in a dosage form comprising a conjugated estrogen equivalent to 153 μg to 1.55 mg sodium estrone sulfate.

45. The method of claim 42, wherein said composition is in a dosage form comprising a conjugated estrogen equivalent to 60 μg to 775 μg sodium equilin sulfate.

46. The method of claim 42, wherein said composition is in a dosage form comprising a conjugated estrogen equivalent to 1.5 μg to 50 μg sodium 17β-estradiol sulfate.

47. The method of claim 42, wherein said composition is administered vaginally once daily for at least 2 consecutive days.

48. (canceled)

49. The method of claim 42, wherein said composition is administered vaginally at least twice per week for at least 1 week.

50. (canceled)

51. (canceled)

52. A method of delivering the vaginal cream composition of claim 1 to a patient in need thereof, said method comprising:
(a) registering in a computer readable storage medium identity of a physician permitted to prescribe said composition;
(b) providing said patient with counseling information concerning a risk attendant to said composition;
(c) obtaining informed consent of said patient to receive said composition despite said risk;
(d) registering said patient in the computer readable medium after obtaining said informed consent; and
(e) permitting said patient access to said vaginal cream composition.

53. The method of claim 52, wherein said access to said vaginal cream composition is a prescription.

54. A method of educating a consumer regarding the composition of claim 1, said method comprising distributing said composition to a consumer with consumer information at a point of sale.

55. The method of claim 54, wherein said consumer information is presented in a format selected from the group consisting of: English language text, a foreign language text, a visual image, a chart, a telephone recording, a website, and access to a live customer service representative.

56. The method of claim 54, wherein said consumer information is a direction for use, appropriate age use, indication, contraindication, appropriate dosing, warning, telephone number or website address.

57. The method of claim 54, further comprising providing professional information to a relevant person in a position to answer a consumer question regarding said vaginal cream composition.

58. (canceled)

59. (canceled)

60. A method of treating a menopausal condition in a female in need thereof, said method comprising vaginally administering a pharmaceutical vaginal cream composition comprising a conjugated estrogen twice per week for at least 2 weeks.

61. The method of claim 60, said method comprising vaginally administering (a) at least once daily for 2 to 13 consecutive days, then (b) twice per week for at least 2 weeks.

62. The method of claim 61, said method comprising vaginally administering (a) at least once daily for 7 consecutive days, then (b) twice per week for at least 2 weeks.

63. The method of claim 60, wherein said menopausal condition is selected from the group consisting of vaginal dryness, pain during intercourse, increased risk of infections, inability to control urination (incontinence), increased frequency of urinary infections, vaginal atrophy, kraurosis vulvae, hot flashes and night sweats, fatigue, emotional changes (mood swings and changes in sexual interest), sleep disturbances (insomnia), drier skin and hair, increased growth of facial and body hair, aches and pains in the joints, headaches, palpitations (rapid, irregular heart beats), vaginal itching, osteoporosis, and generalized itching.

64. (canceled)

65. (canceled)

66. The method of claim 60, wherein said composition is in a dosage form comprising 0.3 mg to 2.5 mg of conjugated estrogens, wherein said conjugated estrogens consist of:
(a) 3.5% to 7.0% by weight sodium 17α-estradiol sulfate;
(b) 10.0% to 19.0% by weight sodium 17α-dihydroequilin sulfate;
(c) 0.5% to 3.5% by weight sodium 17α-dihydroequilin sulfate;
(d) 51.0% to 62.0% by weight sodium estrone sulfate;
(e) 20.0% to 31.0% by weight sodium equilin sulfate;
(f) 0.5% to 2.0% by weight sodium 17β-estradiol sulfate;
(g) 0.2% to 5.0% by weight sodium 17α-dihydroequilenin sulfate;
(h) 0.1% to 1.5% by weight sodium 17β-dihydroequilenin sulfate; and
(i) 0.5% to 6.5% by weight sodium equilenin sulfate;
or an amount of a mixture of conjugated estrogens that provides an estrogenic effect equivalent to that produced by the 0.3 mg to 2.5 mg of conjugated estrogens as defined in (a)-(i).

67. The method of claim 60, wherein said composition is a dosage form comprising a conjugated estrogen equivalent to 153 μg to 1.55 mg sodium estrone sulfate.

68. The method of claim 60, wherein said composition is a dosage form comprising a conjugated estrogen equivalent to 60 μg to 775 μg sodium equilin sulfate.

69. The method of claim 60, wherein said composition is a dosage form comprising a conjugated estrogen equivalent to 1.5 μg to 50 μg sodium 17β-estradiol sulfate.

70. The method of claim 61, wherein 48 hours after the seventh dose during the at least once daily for 7 consecutive days (a), said method provides an average baseline-adjusted estrone Cₘᵢₙ of 2 pg/mL to 17 pg/mL.

71. The method of claim 61, wherein 48 hours after the seventh dose during the at least once daily for 7 consecutive days (a), said method provides an average equilin Cₘᵢₙ of 0 pg/mL to 1 pg/mL.

72. The method of claim 61, wherein 48 hours after the seventh dose during the at least once daily for 7 consecutive days (a), said method provides an average baseline-adjusted 17β-estradiol Cₘᵢₙ of 0 pg/mL to 5 pg/mL.

73. The method of claim 61, wherein 48 hours after the hirteenth dose of the twice per week for at least 2 weeks (b), said method provides an average baseline-adjusted estrone Cₘᵢₙ of 1 pg/mL to 14 pg/mL.

74. The method of claim 61, wherein 48 hours after the thirteenth dose of the twice per week for at least 2 weeks (b), said method provides an average equilin Cₘᵢₙ of 0 pg/mL to 0.5 pg/mL.

75. The method of claim 61, wherein 48 hours after the thirteenth dose of the twice per week for at least 2 weeks (b), said method provides an average baseline-adjusted 17β-estradiol Cₘᵢₙ of 0 pg/mL to 1 pg/mL.

76-88. (canceled)

89. A kit comprising 7 vaginal applicators and a composition comprising a conjugated estrogen.

90-93. (canceled)

94. The kit of claim 89, wherein said composition comprises 0.3 mg to 2.5 mg of conjugated estrogens, wherein said conjugated estrogens consist of:
(a) 3.5% to 7.0% by weight sodium 17α-estradiol sulfate;
(b) 10.0% to 19.0% by weight sodium 17α-dihydroequilin sulfate;
(c) 0.5% to 3.5% by weight sodium 17β-dihydroequilin sulfate;
(d) 51.0% to 62.0% by weight sodium estrone sulfate;
(e) 20.0% to 31.0% by weight sodium equilin sulfate;
(f) 0.5% to 2.0% by weight sodium 17β-estradiol sulfate;
(g) 0.2% to 5.0% by weight sodium 17α-dihydroequilenin sulfate;
(h) 0.1% to 1.5% by weight sodium 17β-dihydroequilenin sulfate; and
(i) 0.5% to 6.5% by weight sodium equilenin sulfate;
or an amount of a mixture of conjugated estrogens that provides an estrogenic effect equivalent to that produced by the 0.3 mg to 2.5 mg of conjugated estrogens as defined in (a)-(i).

95. (canceled)

96. (canceled)

97. The kit of claim 89, further comprising printed instructions for use of said kit.

98. The kit of claim 89, further comprising a printed matter describing the use of the composition to treat a menopausal condition, a pre-recorded media device describing the use of the composition for a menopausal condition, or a planner.

99. (canceled)

100. (canceled)
